(19) United States
(12) Patent Application Publication
(10) Pub. No.: US 2011/0237436 A1
(43) Pub. Date: Sep. 29, 2011

(54) Hydrate and anhydrous crystal form of the sodium salt of 2-iodo-N-[(4-methoxy-6-methyl-1,3,5-triazin-2-yl)carbamoyl]benzenesulfonamide, process for preparation thereof and use thereof as herbicides and plant growth regulators

(75) Inventors: Britta OLENIK, Bottrop (DE); Christian WALDRAFF, Bad Vilbel (DE); Arno RATSCHINSKI, Frankfurt (DE); Martin-Holger HINZ, Huckeswagen (DE); Hans-Joachim RESSEL, Hattersheim (DE)

(73) Assignee: BAYER CROPSCIENCE AG, Monheim (DE)

(21) Appl. No.: 13/039,186

(22) Filed: Mar. 2, 2011

Related U.S. Application Data
(60) Provisional application No. 61/311,008, filed on Mar. 5, 2010.

(30) Foreign Application Priority Data
Mar. 4, 2010 (EP) 10155432.7

(51) Int. Cl. A01N 43/66; C07D 251/16; A01P 13/00; A01P 21/00

(52) U.S. Cl. 504/231; 544/211

(57) ABSTRACT

A hydrate and an anhydrous crystal form of the sodium salt of 2-iodo-N-[(4-methoxy-6-methyl-1,3,5-triazin-2-yl)carbamoyl]benzenesulfonamide are described. These thermodynamically stable crystal forms have particular advantages in the stability of suspension formulations.

### CROSS REFERENCE TO RELATED APPLICATIONS

This application claims priority from EP 10155432.7 filed Mar. 4, 2010 and U.S. Provisional Ser. No. 61/311,008, filed Mar. 5, 2010, the contents of which are incorporated herein by reference in their entireties.

### BACKGROUND OF THE INVENTION

1. Field of the Invention

The invention relates to the technical field of crop protection compositions.

2. Description of Related Art

More specifically, it relates to hydrates and to an anhydrous crystal form of the sodium salt of 2-iodo-N-[(4-methoxy-6-methyl-1,3,5-triazin-2-yl)carbamoyl]benzene-sulfonamide of the formula (I)

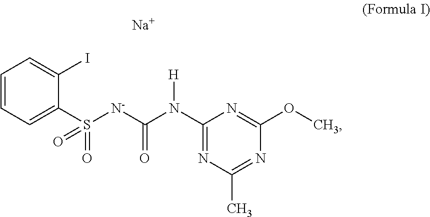

to processes for preparation thereof, and to the use thereof as herbicides and plant growth regulators.

It is known that some organic compounds can occur in only one crystal structure, others (known as polymorphs) in different crystal structures; see, for example, J. Bernstein, R. J. Davey, J. O. Henck, Angew. Chem. Int. Ed., 1999, 38, 3440-3461. For example, EP 1 314 724 A1 discloses two crystal structures of the herbicidal active ingredient sulcotrione.

The compound of the formula (I) known, for example, from WO 2009/053058 (compound I-2 in table 1 therein) has herbicidal properties and is suitable for the production of crop protection compositions which can be employed for weed control. However, it has been found that the amorphous compound of the formula (I) preparable according to the disclosure of WO 2009/053058 is only suitable to a limited degree for the production of user-friendly administration forms. User-friendly administration forms are, for example, suspension formulations or solid formulations in which the compound of the formula (I) is present in finely ground, solid form. In practical testing, it has been found that the compound of the formula (I) preparable according to the disclosure of WO 2009/053058 in suspension formulations can lead to physically unstable formulations. There may, for example, be precipitation, crystal growth or solidification, such that the suspension formulation becomes unusable. These effects can occur spontaneously or over a longer period and cannot be foreseen.

### SUMMARY OF THE INVENTION

It was therefore an object of the present invention to provide a suitable solid form of the compound of the formula (I) specified in WO 2009/053058 which overcomes these disadvantages and is suitable for the production of a suspension formulation which is storage-stable over a prolonged period.

A hydrate and an anhydrous crystal form of the compound of the formula (I) have been found, which do not have the abovementioned disadvantages and are therefore particularly suitable for the production of suspension formulations such as suspension concentrates (SC), suspoemulsions (SE) and oil dispersions (OD), and for solid formulations such as granules (GR, SG and WG) and powders (SP and WP) [CropLife international; Technical Monograph no° 2, 6^{th} edition; Catalogue of pesticide formulation types and international coding system].

The invention therefore provides a hydrate of the sodium salt of 2-iodo-N-[(4-methoxy-6-methyl-1,3,5-triazin-2-yl)carbamoyl]benzenesulfonamide, and an anhydrous form of this sodium salt of 2-iodo-N-[(4-methoxy-6-methyl-1,3,5-triazin-2-yl)carbamoyl]benzenesulfonamide.

### BRIEF DESCRIPTION OF THE DRAWINGS

FIGS. 1-4 depict various embodiments of the instant disclosure.

### DETAILED DESCRIPTION OF A PREFERRED EMBODIMENT

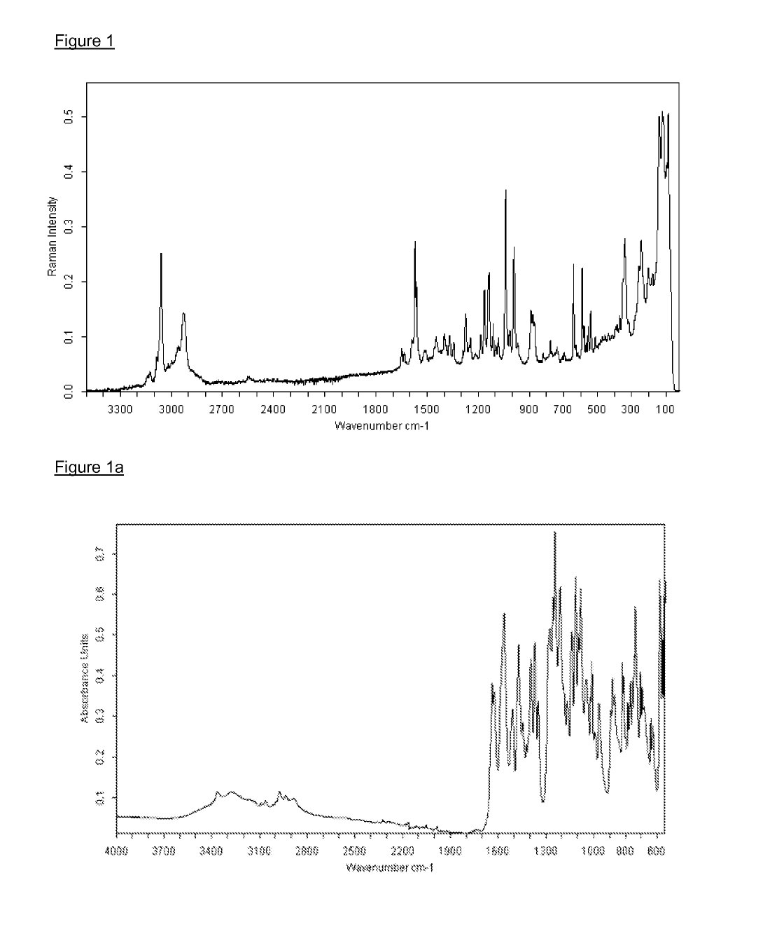

The anhydrous crystal form has a characteristic Raman spectrum (FIG. 1) and a characteristic infrared spectrum (FIG. 1a). This anhydrous crystal form is also referred to hereinafter as “crystal form I”.

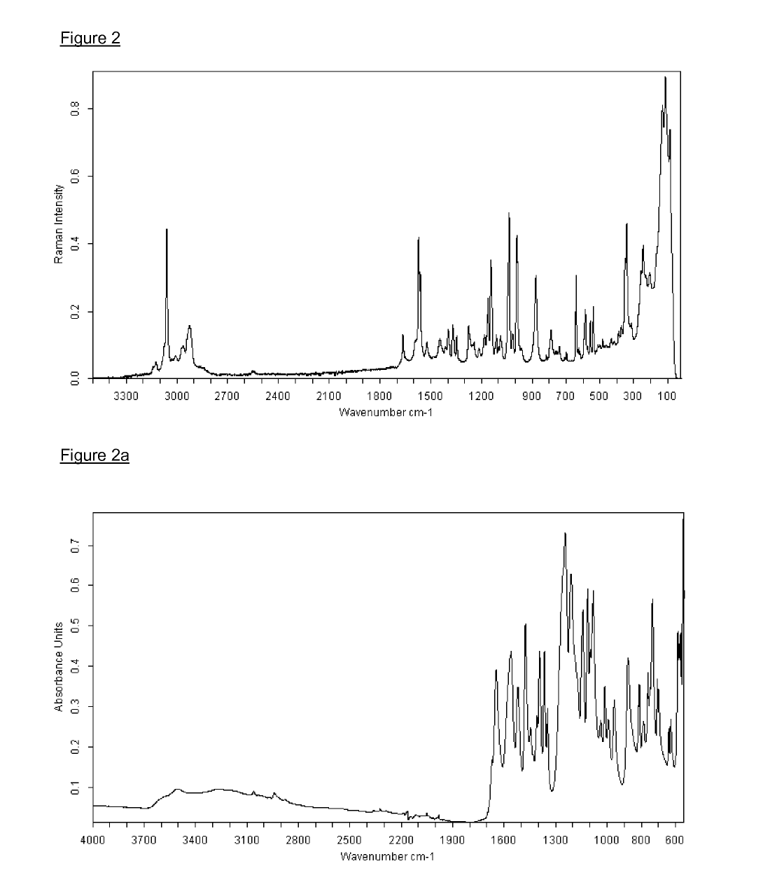

The hydrate of the sodium salt of 2-iodo-N-[(4-methoxy-6-methyl-1,3,5-triazin-2-yl)carbamoyl]benzenesulfonamide (crystal form II) has a characteristic Raman spectrum (FIG. 2) and a characteristic infrared spectrum (FIG. 2a). This crystal form II is also referred to hereinafter as “hydrate”.

The x-ray powder diffractogram of the crystal form I of the sodium salt of 2-iodo-N-[(4-methoxy-6-methyl-1,3,5-triazin-2-yl)carbamoyl]benzenesulfonamide exhibits signal sequences characteristic of this crystal form, which are reported in table 3.

The x-ray powder diffractogram of the hydrate of the sodium salt of 2-iodo-N-[(4-methoxy-6-methyl-1,3,5-triazin-2-yl)carbamoyl]benzenesulfonamide exhibits signal sequences characteristic of this crystal form, which are reported in table 4.

### DESCRIPTION OF THE FIGURES

FIG. 1 shows the Raman spectrum of the anhydrous crystal form I of the sodium salt of 2-iodo-N-[(4-methoxy-6-methyl-1,3,5-triazin-2-yl)carbamoyl]benzenesulfonamide. The values of the band maxima in wavenumbers are listed in table 1.

FIG. 1a shows the infrared spectrum of the crystal form I of the sodium salt of 2-iodo-N-[(4-methoxy-6-methyl-1,3,5-triazin-2-yl)carbamoyl]benzenesulfonamide. The values of the band maxima in wavenumbers are listed in table 1a.

FIG. 2 shows the Raman spectrum of the hydrate of the sodium salt of 2-iodo-N-[(4-methoxy-6-methyl-1,3,5-triazin-2-yl)carbamoyl]benzenesulfonamide. The values of the band maxima in wavenumbers are listed in table 2.

FIG. 2a shows the infrared spectrum of the hydrate of the sodium salt of 2-iodo-N-[(4-methoxy-6-methyl-1,3,5-triazin-2-yl)carbamoyl]benzenesulfonamide. The values of the band maxima in wavenumbers are listed in table 2a.

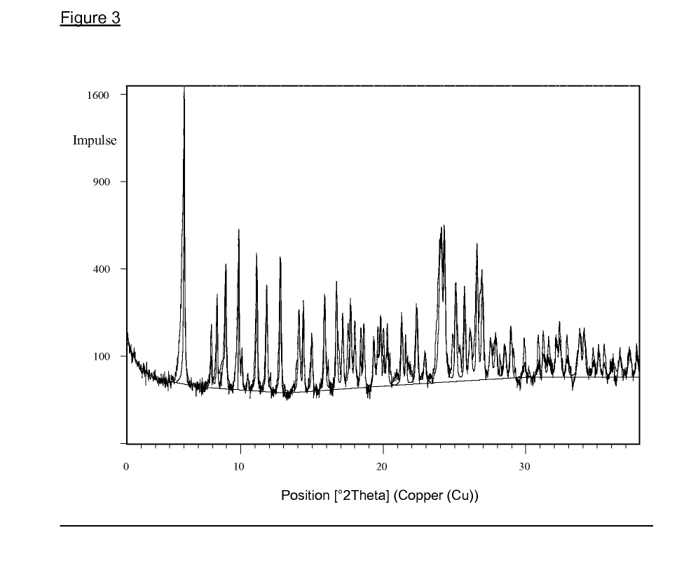

FIG. 3 shows the x-ray powder diffractogram of the crystal form I of the sodium salt of 2-iodo-N-[(4-methoxy-6-methyl-1,3,5-triazin-2-yl)carbamoyl]benzenesulfonamide. The values of the reflections are listed in table 3.

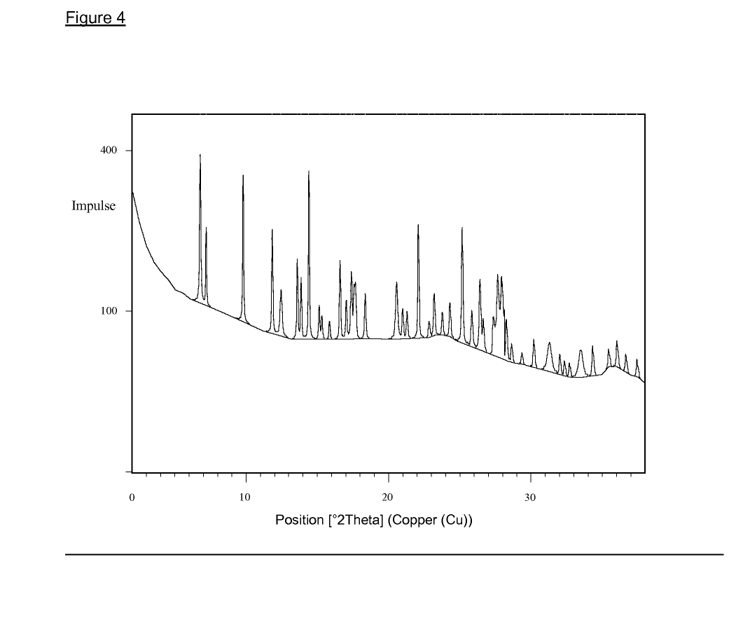

FIG. 4 shows the x-ray powder diffractogram of the hydrate of the sodium salt of 2-iodo-N-[(4-methoxy-6-methyl-1,3,5-triazin-2-yl)carbamoyl]benzenesulfonamide. The values of the reflections are listed in table 4.

TABLE 1
Band maxima of the Raman spectra in [cm⁻¹ ± 3]
Crystal form I

3127
3060
2930
1650
1633
1571
1562
1517
1446
1397
1368
1342
1273
1245
1186
1163
1136
1113
1096
1081
1040
1017
991
891
879
870
776
737
696
589
579
567
554
540
513
437
371
341
254
245
203
176
137
120

TABLE 1a
Band maxima of the infrared spectra in [cm⁻¹ ± 3]
Crystal form I

3364
1637
1624
1562
1508
1471
1445
1395
1369
1348
1279
1254
1243
1209
1166
1136
1113
1096
1080
1037
1011
992
966
892
880
868
818
811
785
767
754
737
705
695
640
627
584
579
568
553

TABLE 2
Band maxima of the Raman spectra in [cm⁻¹ ± 3]
Hydrate

3125
3060
3006
2962
2925
1664
1572
1562
1522
1445
1396
1370
1345
1276
1183
1162
1145
1113
1087
1036
1016
992
879
788
740
641
587
556
539
388
373
352
342
244
205
131
112

TABLE 2a
Band maxima of the infrared spectra in [cm⁻¹ ± 3]
Hydrate

1648
1560
1519
1474
1446
1392
1365
1346
1242
1208
1140
1113
1080
1036
1012
992
957
877
811
787
761
735
706
698
640
628
586
572
554

TABLE 3
Reflections of the x-ray powder diffractogram of the crystal form I of the
sodium salt of 2-iodo-N-[(4-methoxy-6-methyl-1,3,5-triazin-2-
yl)carbamoyl]benzenesulfonamide

| 2 θ | D [Å ± 0.2] |
|---|---|
| 6.0 | 14.7 |
| 8.9 | 9.9 |
| 9.9 | 9.0 |
| 11.1 | 8.0 |
| 12.8 | 6.9 |
| 16.7 | 5.3 |
| 24.0 | 3.7 |
| 24.3 | 3.7 |
| 26.6 | 3.4 |
| 26.9 | 3.3 |

TABLE 4
Reflections of the x-ray powder diffractogram of the hydrate of the
sodium salt of 2-iodo-N-[(4-methoxy-6-methyl-1,3,5-triazin-2-
yl)carbamoyl]benzenesulfonamide

| 2 θ | D [Å ± 0.2] |
|---|---|
| 6.8 | 13.0 |
| 7.2 | 12.3 |
| 9.8 | 9.0 |
| 11.8 | 7.5 |
| 13.6 | 6.5 |
| 14.4 | 6.2 |
| 16.6 | 5.3 |
| 22.1 | 4.0 |
| 25.1 | 3.5 |
| 26.4 | 3.4 |

To determine the Raman spectra, an RFS 100/S FT-Raman from Bruker was used to record at least one spectrum with 128 scans for each component. The infrared spectra were recorded with an FT-IR spectrometer (Bruker Tensor 37) with 64 scans each time. The x-ray powder diffractograms were measured with an XPERT-PRO system with a Cu anode at 25° C.

### Production

The sodium salt of 2-iodo-N-[(4-methoxy-6-methyl-1,3,5-triazin-2-yl)carbamoyl]-benzenesulfonamide itself can be prepared, for example, by one of the processes specified in WO 2009/053058. Depending on the solvent used in the last stage of the process (salt formation), the concentration thereof and the temperature regime, the sodium salt of 2-iodo-N-[(4-methoxy-6-methyl-1,3,5-triazin-2-yl)carbamoyl]benzenesulfonamide crystallizes as a mixture, as a pure amorphous form, or as a hydrate.

The crystal form I of the sodium salt of 2-iodo-N-[(4-methoxy-6-methyl-1,3,5-triazin-2-yl)carbamoyl]benzenesulfonamide can generally be prepared by suspending and/or dissolving the amorphous form of the sodium salt of 2-iodo-N-[(4-methoxy-6-methyl-1,3,5-triazin-2-yl)carbamoyl]benzenesulfonamide or mixtures thereof, obtainable according to WO 2009/053058, in a suitable solvent and treating it at temperatures of 0° C. to 80° C. until quantitative conversion to the crystal form I.

The invention therefore further provides a process for preparing the crystal form I of the sodium salt of 2-iodo-N-[(4-methoxy-6-methyl-1,3,5-triazin-2-yl)carbamoyl]benzenesulfonamide, wherein the amorphous form of the sodium salt of 2-iodo-N-[(4-methoxy-6-methyl-1,3,5-triazin-2-yl)carbamoyl]benzenesulfonamide or mixtures thereof is suspended and/or dissolved in solvents and treated at temperatures of 0° C. to 80° C. until quantitative conversion to the crystal form I.

Suitable solvents which can be used in this process are, for example, lower alcohols such as methanol, ethanol, 2-propanol, or ketones such as acetone, 2-butanone, which can also be used in a mixture with water. Lower alcohols or ketones refer here to those compounds which have one to ten carbon atoms, preferably one to five carbon atoms. Further suitable solvents are benzene, toluene and chlorobenzene.

The conversion to the crystal form I is effected at temperatures less than 100° C., preferably at temperatures of 0° C. to 80° C., more preferably at temperatures of 30° C. to 80° C., most preferably at temperatures of 50° C. to 80° C. The duration of the treatment depends on the temperature and type of solvent. In addition, the duration of the conversion depends on whether seed crystals of the crystal form I are used. The cooling to room temperature is effected preferably with a cooling rate of less than 25° C., more preferably with a cooling rate of less than 20° C. The conversion of a suspension of the amorphous sodium salt of 2-iodo-N-[(4-methoxy-6-methyl-1,3,5-triazin-2-yl)carbamoyl]benzenesulfonamide can generally be brought about without the use of seed crystals within a period of 14 days. When seed crystals of the crystal form I are used in the conversion of a suspension, a treatment time of at least 5 hours is generally sufficient, and in the case of use of greater amounts a treatment time of 24 to 48 hours, in order to achieve a quantitative conversion of the crystals to the crystal form I.

The resulting crystals of the crystal form I are finally removed and the solvent is removed by drying to constant weight at room temperature or elevated temperature.

The crystal form I, due to its stability, is outstandingly suitable for the preparation of formulations, especially suspension formulations, of crop protection compositions. The invention therefore also provides crop protection compositions which comprise the crystal form I of the sodium salt of 2-iodo-N-[(4-methoxy-6-methyl-1,3,5-triazin-2-yl)carbamoyl]benzenesulfonamide alone or in a mixture with assistants and carriers, or else in a mixture with other active ingredients. The invention also includes mixtures of the crystal form I of the sodium salt of 2-iodo-N-[(4-methoxy-6-methyl-1,3,5-triazin-2-yl)carbamoyl]benzenesulfonamide with amorphous sodium salt of 2-iodo-N-[(4-methoxy-6-methyl-1,3,5-triazin-2-yl)carbamoyl]benzenesulfonamide, for example those which occur anywhere in the inventive conversion process of the amorphous sodium salt of 2-iodo-N-[(4-methoxy-6-methyl-1,3,5-triazin-2-yl)carbamoyl]benzenesulfonamide or mixtures thereof to the crystal form I. Preference is given to an active ingredient quality with more than 20% by weight of the crystal form I of the sodium salt of 2-iodo-N-[(4-methoxy-6-methyl-1,3,5-triazin-2-yl)carbamoyl]benzenesulfonamide, more preferably with more than 90% by weight, even more preferably with more than 95% by weight and most preferably with more than 98% by weight.

Optionally, the sodium salt of 2-iodo-N-[(4-methoxy-6-methyl-1,3,5-triazin-2-yl)carbamoyl]benzenesulfonamide in the crystal form I is mixed with one or more other herbicides. Such mixtures also profit from the advantageous properties of the inventive crystal form I.

Due to its stability, the crystal form I of the sodium salt of 2-iodo-N-[(4-methoxy-6-methyl-1,3,5-triazin-2-yl)carbamoyl]benzenesulfonamide is quite generally suitable as a starting material for the production of any crop protection formulations comprising the sodium salt of 2-iodo-N-[(4-methoxy-6-methyl-1,3,5-triazin-2-yl)carbamoyl]benzenesulfonamide, even when the sodium salt of 2-iodo-N-[(4-methoxy-6-methyl-1,3,5-triazin-2-yl)carbamoyl]benzenesulfonamide after the formulation is no longer present in this form, but rather, for instance, in dissolved form.

The invention therefore also provides processes for producing crop protection formulations comprising the sodium salt of 2-iodo-N-[(4-methoxy-6-methyl-1,3,5-triazin-2-yl)carbamoyl]benzenesulfonamide, which use the crystal form I of the sodium salt of 2-iodo-N-[(4-methoxy-6-methyl-1,3,5-triazin-2-yl)carbamoyl]benzene-sulfonamide, and crop protection formulations comprising the sodium salt of 2-iodo-N-[(4-methoxy-6-methyl-1,3,5-triazin-2-yl)carbamoyl]benzenesulfonamide which have been obtained from the crystal form I of the sodium salt of 2-iodo-N-[(4-methoxy-6-methyl-1,3,5-triazin-2-yl)carbamoyl]benzenesulfonamide. The use of the crystal form I increases certainty for formulations of the sodium salt of 2-iodo-N-[(4-methoxy-6-methyl-1,3,5-triazin-2-yl)carbamoyl]benzenesulfonamide and thus reduces the risk of incorrect dosages.

The invention further provides a process for preparing the hydrate of the sodium salt of 2-iodo-N-[(4-methoxy-6-methyl-1,3,5-triazin-2-yl)carbamoyl]benzenesulfonamide, wherein the amorphous form of the sodium salt of 2-iodo-N-[(4-methoxy-6-methyl-1,3,5-triazin-2-yl)carbamoyl]benzenesulfonamide is suspended in mixtures of acetonitrile and water, preferably acetonitrile/water (95:5% by vol.) and treated at temperatures of 5° C. to 60° C. until quantitative conversion to the hydrate.

Likewise suitable solvents which can be used in this process are water-miscible solvents, for example lower alcohols such as methanol, ethanol, 2-propanol, or ketones such as acetone, 2-butanone and methyl isobutyl ketone.

The resulting crystals of the hydrate are finally removed and the solvent is removed by drying to constant weight at room temperature or elevated temperature.

The conversion of a suspension of the amorphous sodium salt of 2-iodo-N-[(4-methoxy-6-methyl-1,3,5-triazin-2-yl)carbamoyl]benzenesulfonamide can generally be brought about without the use of seed crystals within a period of 2-10 hours. When seed crystals of the hydrate are used in the conversion of a suspension, the conversion can be accelerated further.

The inventive hydrate, due to its stability, is likewise outstandingly suitable for the preparation of formulations, especially suspension formulations, of crop protection compositions. The invention therefore also provides crop protection compositions which comprise the inventive hydrate of the sodium salt of 2-iodo-N-[(4-methoxy-6-methyl-1,3,5-triazin-2-yl)carbamoyl]benzenesulfonamide alone or in a mixture with assistants and carriers, or else in a mixture with other active ingredients. The invention also includes mixtures of the inventive hydrate of the sodium salt of 2-iodo-N-[(4-methoxy-6-methyl-1,3,5-triazin-2-yl)carbamoyl]benzenesulfonamide with amorphous sodium salt of 2-iodo-N-[(4-methoxy-6-methyl-1,3,5-triazin-2-yl)carbamoyl]benzenesulfonamide, for example those which occur anywhere in the inventive conversion process of the amorphous sodium salt of 2-iodo-N-[(4-methoxy-6-methyl-1,3,5-triazin-2-yl)carbamoyl]benzenesulfonamide or mixtures thereof to the inventive hydrate.

Preference is given to an active ingredient quality with more than 20% by weight of the inventive hydrate of the sodium salt of 2-iodo-N-[(4-methoxy-6-methyl-1,3,5-triazin-2-yl)carbamoyl]benzenesulfonamide, more preferably with more than 90% by weight, even more preferably with more than 95% by weight and most preferably with more than 98% by weight.

Optionally, the sodium salt of 2-iodo-N-[(4-methoxy-6-methyl-1,3,5-triazin-2-yl)carbamoyl]benzenesulfonamide as the hydrate is mixed with one or more other herbicides. Such mixtures also profit from the advantageous properties of the inventive hydrate.

Due to its stability, the inventive hydrate of the sodium salt of 2-iodo-N-[(4-methoxy-6-methyl-1,3,5-triazin-2-yl)carbamoyl]benzenesulfonamide is quite generally suitable as a starting material for the production of any crop protection formulations comprising the sodium salt of 2-iodo-N-[(4-methoxy-6-methyl-1,3,5-triazin-2-yl)carbamoyl]benzenesulfonamide, even when the sodium salt of 2-iodo-N-[(4-methoxy-6-methyl-1,3,5-triazin-2-yl)carbamoyl]benzenesulfonamide after the formulation is no longer present in this form, but rather, for instance, in dissolved form.

The invention therefore also provides processes for producing crop protection formulations comprising the sodium salt of 2-iodo-N-[(4-methoxy-6-methyl-1,3,5-triazin-2-yl)carbamoyl]benzenesulfonamide, which use the inventive hydrate of the sodium salt of 2-iodo-N-[(4-methoxy-6-methyl-1,3,5-triazin-2-yl)carbamoyl]benzene-sulfonamide, and crop protection formulations comprising the sodium salt of 2-iodo-N-[(4-methoxy-6-methyl-1,3,5-triazin-2-yl)carbamoyl]benzenesulfonamide which have been obtained from the inventive hydrate of the sodium salt of 2-iodo-N-[(4-methoxy-6-methyl-1,3,5-triazin-2-yl)carbamoyl]benzenesulfonamide. The use of the inventive hydrate increases certainty for formulations of the sodium salt of 2-iodo-N-[(4-methoxy-6-methyl-1,3,5-triazin-2-yl)carbamoyl]benzenesulfonamide and thus reduces the risk of incorrect dosages.

The hydrate and the crystal form I of the sodium salt of 2-iodo-N-[(4-methoxy-6-methyl-1,3,5-triazin-2-yl)carbamoyl]benzenesulfonamide can be converted in a known manner to the customary formulations, such as suspension concentrates, colloidal concentrates, dispersible concentrates, emulsifiable concentrates (emulsion concentrates), seed-dressing emulsions, seed-dressing suspensions, granules, microgranules, suspoemulsions, oil dispersions, water-soluble granules, water-soluble concentrates and water-dispersible granules, using suitable assistants and carriers or solvents. In this context, the active compound should be present in a concentration of about 0.5 to 90% by weight of the overall mixture, i.e. in amounts which are sufficient to achieve the dosage level necessary. The formulations are produced, for example, by extending the crystal form I and/or the hydrate of the sodium salt of 2-iodo-N-[(4-methoxy-6-methyl-1,3,5-triazin-2-yl)carbamoyl]benzenesulfonamide with solvents and/or carriers, optionally using emulsifiers and/or dispersants, and/or other assistants, for example penetrants.

Application is effected in the customary manner, by contacting the unwanted plants and/or their habitat with the inventive active ingredient or formulations thereof.

The inventive hydrate and the inventive crystal form I of the sodium salt of 2-iodo-N-[(4-methoxy-6-methyl-1,3,5-triazin-2-yl)carbamoyl]benzenesulfonamide have excellent herbicidal efficacy against a broad spectrum of economically important mono- and dicotyledonous harmful plants. The active ingredients also have good efficacy on perennial weeds which produce shoots from rhizomes, root stocks and other perennial organs and which are difficult to control. It is unimportant whether the substances are applied before sowing, pre-emergence or post-emergence. The invention therefore also provides for the use of the crystal form I and/or of the hydrate of the sodium salt of 2-iodo-N-[(4-methoxy-6-methyl-1,3,5-triazin-2-yl)carbamoyl]benzenesulfonamide for production of a crop protection composition for treatment of weed infestation.

When, for example, the inventive hydrate and/or the inventive crystal form I of the sodium salt of 2-iodo-N-[(4-methoxy-6-methyl-1,3,5-triazin-2-yl)carbamoyl]benzenesulfonamide, especially the hydrate, are applied to the soil surface before germination, either the emergence of the weed seedlings is completely prevented or the weeds grow as far as the cotyledon stage, but then stop growing and finally die off completely after three to four weeks.

On application of the inventive hydrate and/or the inventive crystal form I of the sodium salt of 2-iodo-N-[(4-methoxy-6-methyl-1,3,5-triazin-2-yl)carbamoyl]benzene-sulfonamide post-emergence to the green plant parts, there is likewise very rapid and marked stoppage of growth after the treatment, and the weed plants remain at the growth stage reached at the time of application or die off completely after a certain time, such that weed competition, which is harmful to the crop plants, is thus eliminated very early and in a sustained manner.

Even though the inventive hydrate and the crystal form I of the sodium salt of 2-iodo-N-[(4-methoxy-6-methyl-1,3,5-triazin-2-yl)carbamoyl]benzenesulfonamide, especially the hydrate, display an outstanding herbicidal activity against mono- and dicotyledonous weeds, crop plants of economically important crops, for example wheat, barley, rye, oats, rice, corn, sugarcane, flax and other plantation crops, are damaged only to an insignificant extent, if at all. For these reasons, the inventive compounds are very suitable for selective control of unwanted plant growth in agriculturally useful plants.

In addition, the inventive hydrate and the crystal form I of the sodium salt of 2-iodo-N-[(4-methoxy-6-methyl-1,3,5-triazin-2-yl)carbamoyl]benzenesulfonamide, especially the hydrate, have very advantageous properties with regard to behavior in the environment, especially with regard to rotation behavior, i.e. toward crops which are otherwise sensitive to the inventive hydrate and the crystal form I of the sodium salt of 2-iodo-N-[(4-methoxy-6-methyl-1,3,5-triazin-2-yl)carbamoyl]benzenesulfonamide, for example sugarbeet, sunflower or crucifers, for example oilseed rape, mustard and field mustard.

In addition, the inventive hydrate and the crystal form I of the sodium salt of 2-iodo-N-[(4-methoxy-6-methyl-1,3,5-triazin-2-yl)carbamoyl]benzenesulfonamide, especially the hydrate, have outstanding growth-regulatory properties in crop plants. They intervene in the plant's own metabolism with a regulatory effect, and can thus be used to control plant constituents and to facilitate harvesting, for example by triggering desiccation and stunted growth. In addition, they are also suitable for general control and inhibition of unwanted vegetative growth without killing the plants. Inhibition of vegetative growth is very important for many mono- and dicotyledonous crops, since this can reduce or completely prevent lodging.

Due to their herbicidal and plant growth-regulatory properties, the inventive hydrate and/or the crystal form I of the sodium salt of 2-iodo-N-[(4-methoxy-6-methyl-1,3,5-triazin-2-yl)carbamoyl]benzenesulfonamide can also be used for control of harmful plants in crops of known plants or of genetically modified plants yet to be developed. The transgenic plants are generally notable for particularly advantageous properties, for example resistances to certain pesticides, in particular certain herbicides, resistances to plant diseases or pathogens of plant diseases, such as certain insects, or microorganisms such as fungi, bacteria or viruses. Other particular properties relate, for example, to the harvested material with regard to quantity, quality, storability, composition and specific constituents. For instance, there are known transgenic plants with increased starch content or altered starch quality, or those with a different fatty acid composition of the harvested material.

Similiarly, due to their herbicidal and plant growth-regulatory properties, the inventive hydrate and the crystal form I of the sodium salt of 2-iodo-N-[(4-methoxy-6-methyl-1,3,5-triazin-2-yl)carbamoyl]benzenesulfonamide can also be used for control of harmful plants in crops of known plants or of yet to be developed plants obtained by mutant selection.

Preference is given to the employment of the inventive hydrate and of the crystal form I of the sodium salt of 2-iodo-N-[(4-methoxy-6-methyl-1,3,5-triazin-2-yl)carbamoyl]benzenesulfonamide, especially of the hydrate, in economically important transgenic crops, or crops obtained by mutant selection, of useful and ornamental plants, for example of cereals such as wheat, barley, rye, oats, millet, rice, manioc and corn, or else crops of oilseed rape, potatoes, tomatoes, peas and other vegetable types.

Preferably, the inventive hydrate and the crystal form I of the sodium salt of 2-iodo-N-[(4-methoxy-6-methyl-1,3,5-triazin-2-yl)carbamoyl]benzenesulfonamide, especially the hydrate, can be used as herbicides in useful plant crops which are resistant or have been made resistant by genetic engineering to the phytotoxic effects of the herbicides, or have been obtained by mutant selection. Likewise preferably, the inventive hydrate and the crystal form I of the sodium salt of 2-iodo-N-[(4-methoxy-6-methyl-1,3,5-triazin-2-yl)carbamoyl]benzenesulfonamide, especially the hydrate, can be used as herbicides in useful plant crops which are a crossbreed of plants which have been made resistant by genetic engineering and have been obtained by mutant selection, as described, for example, in WO 2007/024782.

Conventional methods of generating novel plants which have modified properties in comparison to plants occurring to date consist, for example, in traditional breeding methods and the generation of mutants.

It is likewise possible to obtain novel plants with altered properties with the aid of genetic engineering methods (see, for example, EP-A-0221044, EP-A-0131624). For example, several cases of the following have been described:
genetic modifications of crop plants for the purpose of modifying the starch synthesized in the plants (for example WO 92/11376, WO 92/14827, WO 91/19806),
transgenic crop plants which are resistant to particular herbicides of the glufosinate type (cf., for example, EP-A-0242236, EP-A-242246) or glyphosate type (WO 92/00377) or of the sulfonylurea type (EP-A-0257993, U.S. Pat. No. 5,013,659),
transgenic crop plants, for example cotton, with the ability to produce Bacillus thuringiensis toxins (Bt toxins), which make the plants resistant to particular pests (EP-A-0142924, EP-A-0193259),
transgenic crop plants having a modified fatty acid composition (WO 91/13972).

Numerous molecular biology techniques with which novel transgenic plants with modified properties can be produced are known in principle; see, for example, Sambrook et al., 1989, Molecular Cloning, A Laboratory Manual, 2nd edition, Cold Spring Harbor Laboratory Press, Cold Spring Harbor, N.Y.; or Winnacker “Gene and Klone” [“Genes and Clones”], VCH Weinheim 2nd edition 1996 or Christou, “Trends in Plant Science” 1 (1996) 423-431.

For such recombinant manipulations, nucleic acid molecules which allow mutagenesis or sequence alteration by recombination of DNA sequences can be introduced into plasmids. With the aid of the abovementioned standard methods, it is possible, for example, to undertake base exchanges, to remove subsequences, or to add natural or synthetic sequences. For the linkage of the DNA fragments, adapters or linkers may be added to the fragments.

For example, the production of plant cells with a reduced activity of a gene product can be achieved by expressing at least one corresponding antisense RNA, or a sense RNA to achieve a cosuppression effect, or by expressing at least one suitably constructed ribozyme which specifically cleaves transcripts of the abovementioned gene product. To this end, it is firstly possible to use DNA molecules which encompass the entire coding sequence of a gene product including any flanking sequences which may be present, or else DNA molecules which only encompass portions of the coding sequence, in which case these portions must be long enough to have an antisense effect in the cells. It is also possible to use DNA sequences which have a high degree of homology to the coding sequences of a gene product, but are not completely identical. When expressing nucleic acid molecules in plants, the protein synthesized can be localized in any desired compartment of the plant cell. However, in order to achieve localization in a particular compartment, it is possible, for example, to join the coding region to DNA sequences which ensure localization in a particular compartment. Such sequences are known to those skilled in the art (see, for example, Braun et al., EMBO J. 11 (1992), 3219-3227; Wolter et al., Proc. Natl. Acad. Sci. USA 85 (1988), 846-850; Sonnewald et al., Plant J. 1 (1991), 95-106).

The transgenic plant cells can be regenerated by known techniques to give rise to entire plants. In principle, the transgenic plants may be plants of any desired plant species, i.e. either monocotyledonous or dicotyledonous plants.

Thus, it is possible to obtain transgenic plants whose properties are altered by overexpression, suppression or inhibition of homologous (=natural) genes or gene sequences, or expression of heterologous (=foreign) genes or gene sequences.

Preferably, the inventive hydrate and/or the crystal form I of the sodium salt of 2-iodo-N-[(4-methoxy-6-methyl-1,3,5-triazin-2-yl)carbamoyl]benzenesulfonamide can be used in transgenic crops or those obtained by mutant selection, or crosses/hybrids thereof, which are resistant to herbicides from the group of the sulfonylureas, glufosinate-ammonium or glyphosate-isopropylammonium and analogous active ingredients.

On employment of the inventive hydrate and/or of the crystal form I of the sodium salt of 2-iodo-N-[(4-methoxy-6-methyl-1,3,5-triazin-2-yl)carbamoyl]benzenesulfon-amide in transgenic crops or those obtained by mutant selection or crosses thereof, not only do the effects toward harmful plants observed in other crops occur, but often also effects which are specific to application in the particular transgenic crops or those obtained by mutant selection or crosses thereof, for example an altered or specifically widened weed spectrum which can be controlled, altered application rates which can be used for the application, preferably good combinability with the herbicides against which the transgenic crop is resistant, and influencing of growth and yield of the transgenic crop plants.

The invention therefore also provides for the use of the inventive hydrate and of the crystal form I of the sodium salt of 2-iodo-N-[(4-methoxy-6-methyl-1,3,5-triazin-2-yl)carbamoyl]benzenesulfonamide, especially the hydrate, as herbicides for control of harmful plants in transgenic crop plants or those obtained by mutant selection or crosses thereof.

The inventive hydrate and/or the crystal form I of the sodium salt of 2-iodo-N-[(4-methoxy-6-methyl-1,3,5-triazin-2-yl)carbamoyl]benzenesulfonamide can be employed in the form of spray powders, emulsifiable concentrates, sprayable solutions, dusting products or granules, in the customary formulations. The invention therefore also provides herbicidal and plant growth-regulating compositions which comprise the inventive hydrate and the crystal form I of the sodium salt of 2-iodo-N-[(4-methoxy-6-methyl-1,3,5-triazin-2-yl)carbamoyl]benzenesulfonamide.

The inventive hydrate and the anhydrous crystal polymorph I of the sodium salt of 2-iodo-N-[(4-methoxy-6-methyl-1,3,5-triazin-2-yl)carbamoyl]benzenesulfonamide, especially the hydrate, can be formulated in various ways, according to which biological and/or physicochemical parameters are defined. Examples of possible formulations include: wettable powders (WP), water-soluble powders (SP), water-soluble concentrates, emulsifiable concentrates (EC), emulsions (EW), such as oil-in-water and water-in-oil emulsions, sprayable solutions, suspension concentrates (SC), oil- or water-based dispersions, oil-miscible solutions, capsule suspensions (CS), dusting products (DP), seed-dressing products, granules for broadcasting and soil application, granules (GR) in the form of microgranules, sprayable granules, coated granules and adsorption granules, water-dispersible granules (WG), water-soluble granules (SG), ULV formulations, microcapsules and waxes.

These individual formulation types are known in principle and are described, for example, in: Winnacker-Küchler, “Chemische Technologie” [Chemical Technology], volume 7, C. Hanser Verlag Munich, 4th edition 1986, Wade van Valkenburg, “Pesticide Formulations”, Marcel Dekker, N.Y., 1973; K. Martens, “Spray Drying” Handbook, 3rd ed. 1979, G. Goodwin Ltd. London.

The necessary formulation assistants, such as inert materials, surfactants, solvents and further additives are likewise known and are described, for example, in: Watkins, “Handbook of Insecticide Dust Diluents and Carriers”, 2nd ed., Darland Books, Caldwell N.J., H. v. Olphen, “Introduction to Clay Colloid Chemistry”; 2nd ed., J. Wiley & Sons, N.Y.; C. Marsden, “Solvents Guide”; 2nd ed., Interscience, N.Y. 1963; McCutcheon's “Detergents and Emulsifiers Annual”, MC Publ. Corp., Ridgewood N.J.; Sisley and Wood, “Encyclopedia of Surface Active Agents”, Chem. Publ. Co. Inc., N.Y. 1964; Schönfeldt, “Grenzflächenaktive Äthylenoxidaddukte” [Interface-active Ethylene Oxide Adducts], Wiss. Verlagsgesell., Stuttgart 1976; Winnacker-Küchler, “Chemische Technologie”, volume 7, C. Hanser Verlag Munich, 4th ed. 1986.

On the basis of these formulations, it is also possible to produce combinations of the inventive hydrate and/or of the crystal form I of the sodium salt of 2-iodo-N-[(4-methoxy-6-methyl-1,3,5-triazin-2-yl)carbamoyl]benzenesulfonamide with other pesticidally active substances, for example insecticides, acaricides, herbicides, fungicides, and with safeners, fertilizers and/or growth regulators, for example in the form of a finished formulation or as a tankmix.

Wettable powders are preparations which can be dispersed uniformly in water and, in addition to the active ingredient, apart from a diluent or inert substance, also comprise surfactants of the ionic and/or nonionic type (wetting agents, dispersants), for example polyoxyethylated alkylphenols, polyoxyethylated fatty alcohols, polyoxyethylated fatty amines, fatty alcohol polyglycol ether sulfates, alkanesulfonates, alkylbenzenesulfonates, sodium lignosulfonate, sodium 2,2′-dinaphthylmethane-6,6′-disulfonate, sodium dibutylnaphthalenesulfonate or else sodium oleoylmethyltaurinate. To produce the wettable powders, the herbicidally active ingredients are ground finely, for example in customary apparatus such as hammer mills, blower mills and air-jet mills, and simultaneously or subsequently mixed with the formulation assistants. Emulsifiable concentrates are produced by dissolving the active ingredient in an organic solvent, for example butanol, cyclohexanone, dimethylformamide, xylene or else relatively high-boiling aromatics or hydrocarbons or mixtures of the organic solvents, with addition of one or more surfactants of the ionic and/or nonionic type (emulsifiers). The emulsifiers used may, for example, be: ionic emulsifiers such as calcium alkylarylsulfonate salts, for example calcium dodecylbenzenesulfonate or sodium bisalkylsulfosuccinates, or nonionic emulsifiers such as fatty acid polyglycol esters, alkylaryl polyglycol ethers, fatty alcohol polyglycol ethers, propylene oxide-ethylene oxide condensation products, alkyl polyethers, sorbitan esters, for example sorbitan fatty acid esters, or polyoxyethylene sorbitan esters, for example polyoxyethylene sorbitan fatty acid esters.

Dusting products are obtained by grinding the active ingredient with finely distributed solid substances, for example talc, natural clays, such as kaolin, bentonite and pyrophyllite, or diatomaceous earth.

Suspension concentrates may be water- or oil-based. They can be produced, for example, by wet grinding by means of commercial bead mills with optional addition of surfactants as already listed above, for example, for the other formulation types.

Emulsions, for example oil-in-water emulsions (EW), can be produced, for example, by means of stirrers, colloid mills and/or static mixers using aqueous organic solvents and optionally surfactants as already listed above, for example, for the other formulation types.

Granules can be produced either by spraying the active ingredient onto granular inert material capable of adsorption or by applying active ingredient concentrates to the surface of carrier substances, such as sand, kaolinites or granular inert material, by means of adhesives, for example polyvinyl alcohol, sodium polyacrylate or mineral oils. Suitable active ingredients can also be granulated in the manner customary for the production of fertilizer granules—if desired as a mixture with fertilizers.

Water-dispersible granules are produced generally by the customary processes such as spray-drying, fluidized bed granulation, pan granulation, mixing with high-speed mixers and extrusion without solid inert material.

For the production of pan granules, fluidized bed granules, extruder granules and spray granules, see, for example, processes in “Spray-Drying Handbook” 3rd ed. 1979, G. Goodwin Ltd., London; J. E. Browning, “Agglomeration”, Chemical and Engineering 1967, pages 147 ff.; “Perry's Chemical Engineer's Handbook”, 5th ed., McGraw-Hill, New York 1973, p. 8-57.

For further details regarding the formulation of crop protection compositions, see, for example, G. C. Klingman, “Weed Control as a Science”, John Wiley and Sons, Inc., New York, 1961, pages 81-96 and J. D. Freyer, S. A. Evans, “Weed Control Handbook”, 5th ed., Blackwell Scientific Publications, Oxford, 1968, pages 101-103.

The agrochemical formulations contain generally 0.1 to 99% by weight, preferably 0.1 to 95% by weight, especially preferably 0.5 to 90% by weight, of the inventive hydrate and/or of the crystal form I of the sodium salt of 2-iodo-N-[(4-methoxy-6-methyl-1,3,5-triazin-2-yl)carbamoyl]benzenesulfonamide, especially the hydrate of the sodium salt of 2-iodo-N-[(4-methoxy-6-methyl-1,3,5-triazin-2-yl)carbamoyl]-benzenesulfonamide.

In wettable powders, the active ingredient concentration is, for example, from about 10 to 90% by weight, the remainder to 100% by weight consisting of customary formulation components. In the case of emulsifiable concentrates, the active ingredient concentration may be about 1 to 90% and preferably from 5 to 80% by weight. Formulations in the form of dusts contain 1 to 30% by weight of active ingredient, preferably usually 5 to 20% by weight, of active ingredient; sprayable solutions contain about 0.05 to 80% by weight, preferably 2 to 50% by weight, of active ingredient. In the case of water-dispersible granules, the active ingredient content depends partly on whether the active compound is present in liquid or solid form and on which granulation assistants, fillers, etc., are used. In the water-dispersible granules, the content of active ingredient is, for example, between 1 and 95% by weight, preferably between 10 and 80% by weight.

In addition, the active ingredient formulations mentioned optionally comprise the respective customary tackifiers, wetting agents, dispersants, emulsifiers, penetrants, preservatives, antifreeze agents and solvents, fillers, carriers and dyes, defoamers, evaporation inhibitors and agents which influence the pH and the viscosity. If the extender used is water, it is also possible to use, for example, organic solvents as auxiliary solvents. Useful liquid solvents are essentially: aromatics such as xylene, toluene or alkylnaphthalenes, chlorinated aromatics and chlorinated aliphatic hydrocarbons such as chlorobenzenes, chloroethylenes or methylene chloride, aliphatic hydrocarbons such as cyclohexane or paraffins, for example mineral oil fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethylformamide and dimethyl sulfoxide, or else water.

Useful solid carriers include: for example ammonium salts and natural rock flours such as kaolins, clays, talc, chalk, quartz, attapulgite, montmorillonite or diatomaceous earth, and ground synthetic minerals, such as finely divided silica, alumina and silicates. Useful solid carriers for granules are: for example crushed and fractionated natural rocks such as calcite, marble, pumice, sepiolite and dolomite, and also synthetic granules of inorganic and organic meals, and granules of organic material such as sawdust, coconut shells, corn cobs and tobacco stalks. Useful emulsifiers and/or foam formers are: for example nonionic and anionic emulsifiers such as polyoxyethylene fatty acid esters, polyoxyethylene fatty alcohol ethers, for example alkylaryl polyglycol ethers, alkylsulfonates, alkylsulfates, arylsulfonates and protein hydrolyzates. Useful dispersants are: for example lignosulfite waste liquors and methylcellulose.

In the formulations, it is possible to use tackifiers such as carboxymethylcellulose and natural and synthetic polymers in the form of powders, granules or latices, such as gum arabic, polyvinyl alcohol and polyvinyl acetate, as well as natural phospholipids such as cephalins and lecithins, and synthetic phospholipids.

The herbicidal action of the inventive hydrate and/or of the crystal form I of the sodium salt of 2-iodo-N-[(4-methoxy-6-methyl-1,3,5-triazin-2-yl)carbamoyl]benzene-sulfonamide can also be improved, for example, by means of surface-active substances, preferably by means of wetting agents from the group of the fatty alcohol polyglycol ethers. The fatty alcohol polyglycol ethers preferably contain 10-18 carbon atoms in the fatty alcohol radical and 2-20 ethylene oxide units in the polyglycol ether moiety. The fatty alcohol polyglycol ethers may be present in nonionic form, or ionic form, for example in the form of fatty alcohol polyglycol ether sulfates, which may be used, for example, as alkali metal salts (for example sodium salts and potassium salts) or ammonium salts, or else as alkaline earth metal salts, such as magnesium salts, such as C₁₂/C₁₄ fatty alcohol diglycol ether sulfate sodium (Genapol® LRO, Clariant GmbH); see, for example, EP-A-0476555, EP-A-0048436, EP-A-0336151 or U.S. Pat. No. 4,400,196 and also Proc. EWRS Symp. “Factors Affecting Herbicidal Activity and Selectivity”, 227-232 (1988). Nonionic fatty alcohol polyglycol ethers are, for example, (C₁₀-C₁₈), preferably (C₁₀-C₁₄), fatty alcohol polyglycol ethers (for example isotridecyl alcohol polyglycol ethers) containing, for example, 2-20, preferably 3-15, ethylene oxide units, for example those from the Genapol® X-series, such as Genapol® X-030, Genapol® X-060, Genapol® X-080 or Genapol® X-150 (all from Clariant GmbH).

The present invention thus encompasses the combination of the inventive hydrate and/or of the crystal form I of the sodium salt of 2-iodo-N-[(4-methoxy-6-methyl-1,3,5-triazin-2-yl)carbamoyl]benzenesulfonamide with the aforementioned wetting agents from the group of the fatty alcohol polyglycol ethers which preferably contain 10-18 carbon atoms in the fatty alcohol radical and 2-20 ethylene oxide units in the polyglycol ether moiety and may be present in nonionic or ionic form (for example as fatty alcohol polyglycol ether sulfates). Preference is given to C₁₂/C₁₄ fatty alcohol diglycol ether sulfate sodium (Genapol® LRO, Clariant GmbH) and isotridecyl alcohol polyglycol ether having 3-15 ethylene oxide units, for example from the Genapol® X-series, such as Genapol® X-030, Genapol® X-060, Genapol® X-080 and Genapol® X-150 (all from Clariant GmbH). It is also known that fatty alcohol polyglycol ethers, such as nonionic or ionic fatty alcohol polyglycol ethers (for example fatty alcohol polyglycol ether sulfates) are also suitable as penetrants and activity enhancers for a number of other herbicides, including herbicides from the group of the imidazolinones (see, for example, EP-A-0502014).

The herbicidal action of the inventive hydrate and of the inventive crystal form I of the sodium salt of 2-iodo-N-[(4-methoxy-6-methyl-1,3,5-triazin-2-yl)carbamoyl]benzene-sulfonamide, can also be enhanced by the use of vegetable oils. The term vegetable oils is understood to mean oils from oleaginous plant species, such as soybean oil, rapeseed oil, corn oil, sunflower oil, cottonseed oil, linseed oil, coconut oil, palm oil, safflower oil or castor oil, especially rapeseed oil, and also the transesterification products thereof, for example alkyl esters, such as rapeseed oil methyl ester or rapeseed oil ethyl ester.

The vegetable oils are preferably esters of C₁₀-C₂₂, preferably C₁₂-C₂₀, fatty acids. The C₁₀-C₂₂-fatty acid esters are, for example, esters of unsaturated or saturated C₁₀C₂₂-fatty acids, especially those having an even number of carbon atoms, for example erucic acid, lauric acid, palmitic acid and especially C₁₈ fatty acids, such as stearic acid, oleic acid, linoleic acid or linolenic acid.

Examples of C₁₀-C₂₂ fatty acid esters are esters which are obtained by reacting glycerol or glycol with the C₁₀-C₂₂ fatty acids present, for example, in oils from oleaginous plant species, or C₁-C₂₀-alkyl C₁₀-C₂₂ fatty acid esters which can be obtained, for example, by transesterification of the aforementioned glycerol or glycol C₁₀-C₂₂ fatty acid esters with C₁-C₂₀-alcohols (for example methanol, ethanol, propanol or butanol). The transesterification can be effected by known methods, as described, for example, in Römpp Chemie Lexikon, 9th edition, Volume 2, page 1343, Thieme Verlag Stuttgart.

Preferred C₁-C₂₀-alkyl C₁₀-C₂₂ fatty acid esters are methyl esters, ethyl esters, propyl esters, butyl esters, 2-ethylhexyl esters and dodecyl esters. Preferred glycol and glycerol C₁₀-C₂₂ fatty acid esters are the homogeneous or mixed glycol esters and glycerol esters of C₁₀-C₂₂ fatty acids, especially fatty acids having an even number of carbon atoms, for example erucic acid, lauric acid, palmitic acid and especially C₁₈-fatty acids, such as stearic acid, oleic acid, linoleic acid or linolenic acid.

The vegetable oils may be present in combinations with the inventive hydrate and/or the crystal form I of the sodium salt of 2-iodo-N-[(4-methoxy-6-methyl-1,3,5-triazin-2-yl)carbamoyl]benzenesulfonamide, for example in the form of commercially available oil-containing formulation additives, especially those based on rapeseed oil such as Hasten® (Victorian Chemical Company, Australia, referred to hereinafter as Hasten, main constituent: rapeseed oil ethyl ester), Actirob®B (Novance, France, referred to hereinafter as ActirobB, main constituent: rapeseed oil methyl ester), Rako-Binol® (Bayer AG, Germany, referred to hereinafter as Rako-Binol, main constituent: rapeseed oil), Renol® (Stefes, Germany, referred to hereinafter as Renol, vegetable oil constituent: rapeseed oil methyl ester) or Stefes Mero® (Stefes, Germany, referred to hereinafter as Mero, main constituent: rapeseed oil methyl ester).

In a further embodiment, the present invention encompasses combinations of the inventive hydrate and/or the crystal form I of the sodium salt of 2-iodo-N-[(4-methoxy-6-methyl-1,3,5-triazin-2-yl)carbamoyl]benzenesulfonamide with the aforementioned vegetable oils such as rapeseed oil, preferably in the form of commercially available oil-containing formulation additives, especially those based on rapeseed oil such as Hasten® (Victorian Chemical Company, Australia, referred to hereinafter as Hasten, main constituent: rapeseed oil ethyl ester), Actirob®B (Novance, France, referred to hereinafter as ActirobB, main constituent: rapeseed oil methyl ester), Rako-Binol®

(Bayer AG, Germany, referred to hereinafter as Rako-Binol, main constituent: rapeseed oil), Renol® (Stefes, Germany, referred to hereinafter as Renol, vegetable oil constituent: rapeseed oil methyl ester) or Stefes Mero® (Stefes, Germany, referred to hereinafter as Mero, main constituent: rapeseed oil methyl ester).

It is additionally possible to use dyes such as inorganic pigments, for example iron oxide, titanium oxide and Prussian Blue, and organic dyes such as alizarin dyes, azo dyes and metal phthalocyanine dyes, and trace nutrients such as salts of iron, manganese, boron, copper, cobalt, molybdenum and zinc.

The inventive hydrate and/or the crystal form I of the sodium salt of 2-iodo-N-[(4-methoxy-6-methyl-1,3,5-triazin-2-yl)carbamoyl]benzenesulfonamide, especially the hydrate, can be used as such or in combination, in the form of preparations (formulations) thereof with other pesticidally active substances, for example insecticides, acaricides, nematicides, herbicides, fungicides, safeners, fertilizers and/or growth regulators, for example as a finished formulation or as tankmixes.

As combination partners for the inventive hydrate and/or the inventive crystal form I of the sodium salt of 2-iodo-N-[(4-methoxy-6-methyl-1,3,5-triazin-2-yl)carbamoyl]benzenesulfonamide, especially for the hydrate, in mixture formulations or in a tankmix, it is possible to use, for example, known, preferably active herbicidal ingredients based on inhibition of, for example, acetolactate synthase, acetyl coenzyme A carboxylase, PS I, PS II, HPPDO, phytoene desaturase, protoporphyrinogen oxidase, glutamine synthetase, cellulose biosynthesis, 5-enolpyruvylshikimate 3-phosphate synthetase. Such compounds and also other usable compounds, some with an unknown or different mechanism of action are described, for example, in Weed Research 26, 441-445 (1986), or in the handbook “The Pesticide Manual”, 12th edition 2000, or 13th edition 2003 or 14th edition 2006/2007, or in the corresponding “e-Pesticide Manual”, Version 4 (2006), each published by the British Crop Protection Council, (also referred to hereinafter as “PM”), and literature cited therein. Lists of common names are also available in “The Compendium of Pesticide Common Names” on the Internet. Herbicides which are known from the literature and can be combined with the inventive hydrate and/or the crystal form I of the sodium salt of 2-iodo-N-[(4-methoxy-6-methyl-1,3,5-triazin-2-yl)carbamoyl]benzenesulfonamide include, for example, the active ingredients listed below: (N.B.: The herbicides are designated either with the common name according to the International Organization for Standardization (ISO) or with the chemical name, in some cases together with a standard code number, and always refer to all application forms, such as acids, salts, esters and isomers such as stereoisomers, and optical isomers, especially the commercially available form or the standard commercial forms, unless the context suggests anything else. In the case of sulfonamides such as sulfonylureas, salts also include those which result from exchange of a hydrogen atom on the sulfonamide group for a cation. In this case, one or else, in some cases, more than one application form is mentioned.):

acetochlor; acibenzolar-S-methyl; acifluorfen(-sodium); aclonifen; AD-67; AKH 7088, i.e. [[[1-[5-[2-chloro-4-(trifluoromethyl)phenoxy]-2-nitrophenyl]-2-methoxyethylidene]-amino]oxy]acetic acid and -acetic acid methyl ester; alachlor; alloxydim(-sodium); ametryn; amicarbazone, amidochlor, amidosulfuron; aminopyralid; amitrol; ammonium pelargonate; AMS, i.e. ammonium sulfamate; ancimidol; anilofos; asulam; atrazine; aviglycine; azafenidin, azimsulfuron (DPX-A8947); aziprotryn; barban; BAS 516 H, i.e. 5-fluoro-2-phenyl-4H-3,1-benzoxazin-4-one; beflubutamid (UBH-509), benazolin(-ethyl); bencarbazone; benfluralin; benfuresate; bensulfuron(-methyl); bensulide; bentazone; benzfendizone; benzobicyclon, benzofenap; benzofluor; benzoylprop(-ethyl); benzthiazuron; bialaphos; bifenox; bispyribac(-sodium) (KIN-2023); borax; bromacil; bromobutide; bromofenoxim; bromoxynil; bromuron; buminafos; busoxinone; butachlor; butafenacil; butamifos; butenachlor (KH-218); buthidazole; butralin; butroxydim; butylate; cafenstrole (CH-900); caloxydim; carbetamide; carfentrazone(-ethyl); catechin; CDAA, i.e. 2-chloro-N,N-di-2-propenylacetamide; CDEC, i.e. 2-chlorallyl diethyldithiocarbamate; chlormesulon; chlomethoxyfen; chloramben; chlorazifop-butyl; chlorbromuron; chlorbufam; chlorfenac; chlorfenprop; chlorflurecol(-methyl); chlorflurenol(-methyl); chloridazon; chlorimuron(-ethyl); chlormequat(-chloride); chlornitrofen; chlorophthalim (MK-616); chlorotoluron; chloroxuron; chlorpropham; chlorsulfuron; chlorthal-dimethyl; chlorthiamid; chlortoluron; cinidon(-methyl and -ethyl); cinmethylin; cinosulfuron; clefoxydim; clethodim; clodinafop and ester derivatives thereof (e.g. clodinafop-propargyl); clofencet; clomazone; clomeprop; cloprop; cloproxydim; clopyralid; clopyrasulfuron(-methyl); cloransulam(-methyl); cumyluron (JC 940); cyanamide; cyanazine; cycloate; cyclosulfamuron (AC 104); cycloxydim; cycluron; cyhalofop and ester derivatives thereof (e.g. butyl ester, DEH-112); cyperquat; cyprazine; cyprazole; daimuron; 2,4-D; 2,4-DB; dalapon; daminozide; dazomet; n-decanol; desmedipham; desmetryn; di-allate; dicamba; dichlobenil; dichlormid; dichlorprop(—P) salts; diclofop and esters thereof such as diclofop-methyl; diclofop-P(-methyl); diclosulam; diethatyl(-ethyl); difenoxuron; difenzoquat-(-metilsulfate); diflufenican; diflufenzopyr(-sodium); dimefuron; dimepiperate; dimethachlor; dimethametryn; dimethazone; dimethenamid (SAN-582H); dimethenamide-P; dimethylarsinic acid; dimethipin; dimetrasulfuron; dimexyflam; dinitramine; dinoseb; dinoterb; diphenamid; dipropetryn; diquat salts; dithiopyr; diuron; DNOC; eglinazine-ethyl; EL 77, i.e. 5-cyano-1-(1,1-dimethylethyl)-N-methyl-1H-pyrazole-4-carboxamide; endothal; epoprodan; EPTC; esprocarb; ethalfluralin; ethametsulfuron-methyl; ethephon; ethidimuron; ethiozin; ethofumesate; ethoxyfen and esters thereof (e.g. ethyl ester, HN-252); ethoxysulfuron; etobenzanid (HW 52); F5231, i.e. N-[2-chloro-4-fluoro-5-[4-(3-fluoropropyl)-4,5-dihydro-5-oxo-1H-tetrazol-1-yl]phenyl]ethanesulfonamide; fenchlorazole(-ethyl); fenclorim; fenoprop; fenoxan, fenoxaprop and fenoxaprop-P and esters thereof, e.g. fenoxaprop-P-ethyl and fenoxaprop-ethyl; fenoxydim; fentrazamide; fenuron; ferrous sulfate; flamprop(-methyl or -isopropyl or -isopropyl-L); flamprop-M(-methyl or -isopropyl); flazasulfuron; floazulate (JV-485); florasulam; fluazifop and fluazifop-P and esters thereof, e.g. fluazifop-butyl and fluazifop-P-butyl; fluazolate; flucarbazone(-sodium); flucetosulfuron; fluchloralin; flufenacet; flufenpyr(-ethyl); flumetralin; flumetsulam; flumeturon; flumiclorac(-pentyl); flumioxazin (S-482); flumipropyn; fluometuron; fluorochloridone; fluorodifen; fluoroglycofen(-ethyl); flupoxam (KNW-739); flupropacil (UBIC-4243); flupropanoate; flupyrsulfuron(-methyl)(-sodium); flurenol(-butyl); fluridone; fluorochloridone; fluoroxypyr(-meptyl); flurprimidol; flurtamone; fluthiacet(-methyl) (KIN-9201); fluthiamide; fluxofenim; fomesafen; foramsulfuron; forchlorfenuron; fosamine; furyloxyfen; gibberillic acid; glufosinate(-ammonium); glyphosate(-isopropylammonium); halosafen; halosulfuron(-methyl); haloxyfop and esters thereof; haloxyfop-P(═R-haloxyfop) and esters thereof; HC-252; hexazinone; HNPC-C9908, i.e. methyl 2-[[[[[4-methoxy-6-(methylthio)-2-pyrimidinyl]-amino]carbonyl]amino]sulfonyl]benzoate; imazamethabenz(-methyl); imazamox; imazapic; imazapyr; imazaquin and salts such as the ammonium salt; imazethapyr; imazosulfuron; inabenfide; indanofan; iodosulfuron-methyl(-sodium); ioxynil; isocarbamid; isopropalin; isoproturon; isouron; isoxaben; isoxachlortole; isoxaflutole; isoxapyrifop; karbutilate; lactofen; lenacil; linuron; maleic hydrazide (MH); MBTA; MCPA; MCPB; mecoprop(—P); mefenacet; mefluidide; mepiquat(-chloride); mesosulfuron(-methyl); mesotrione; metam; metamifop; metamitron; metazachlor; methabenzthiazuron; metham; methazole; methoxyphenone; methylarsonic acid; methyl-cyclopropene; methyldymron; methylisothiocyanate; methabenzthiazuron; metobenzuron; metobromuron; (alpha-)metolachlor; metosulam (XRD 511); metoxuron; metribuzin; metsulfuron-methyl; molinate; monalide; monocarbamide dihydrogensulfate; monolinuron; monuron; monosulfuron; MT 128, i.e. 6-chloro-N-(3-chloro-2-propenyl)-5-methyl-N-phenyl-3-pyridazineamine; MT 5950, i.e. N-[3-chloro-4-(1-methylethyl)phenyl]-2-methylpentanamide; naproanilide; napropamide; naptalam; NC 310, i.e. 4-(2,4-dichlorobenzoyl)-1-methyl-5-benzyloxypyrazole; neburon; nicosulfuron; nipyraclofen; nitralin; nitrofen; nitrophenolate mixture; nitrofluorfen; nonanoic acid; norflurazon; orbencarb; orthosulfamuron; oxabetrinil; oryzalin; oxadiargyl (RP-020630); oxadiazon; oxasulfuron; oxaziclomefone; oxyfluorfen; paclobutrazol; paraquat(-dichloride); pebulate; pelargonic acid; pendimethalin; penoxulam; pentachlorophenol; pentanochlor; pentoxazone; perfluidone; pethoxamid; phenisopham; phenmedipham; picloram; picolinafen; pinoxaden; piperophos; piributicarb; pirifenop-butyl; pretilachlor; primisulfuron(-methyl); probenazole; procarbazone-(sodium); procyazine; prodiamine; profluralin; profoxydim; prohexadione(-calcium); prohydrojasmon; proglinazine(-ethyl); prometon; prometryn; propachlor; propanil; propaquizafop and esters thereof; propazine; propham; propisochlor; propoxycarbazone(-sodium) (MKH-6561); propyzamide; prosulfalin; prosulfocarb; prosulfuron (CGA-152005); prynachlor; pyraclonil; pyraflufen(-ethyl) (ET-751); pyrasulfotole; pyrazolynate; pyrazon; pyrazosulfuron(-ethyl); pyrazoxyfen; pyribambenz-isopropyl (ZJ 0702); pyrimbambenz-propyl (ZJ 0273); pyribenzoxim; pyributicarb; pyridafol; pyridate; pyriftalid; pyriminobac(-methyl) (KIN-6127); pyrimisulfan (KIN-5996); pyrithiobac-(-sodium) (KIN-2031); pyroxasulfone (KIN-485); pyroxofop and esters thereof (e.g. propargyl ester); pyroxsulam; quinclorac; quinmerac; quinoclamine; quinofop and ester derivatives thereof, quizalofop and quizalofop-P and ester derivatives thereof, e.g. quizalofop-ethyl; quizalofop-P-tefuryl and -ethyl; renriduron; rimsulfuron (DPX-E 9636); S 275, i.e. 2-[4-chloro-2-fluoro-5-(2-propynyloxy)phenyl]-4,5,6,7-tetrahydro-2H-indazole; secbumeton; sethoxydim; siduron; simazine; simetryn; sintofen; SN 106279, i.e. 2-[[7-[2-chloro-4-(trifluoromethyl)phenoxy]-2-naphthalenyl]-oxy]propanoic acid and -propanoic acid methyl ester; sulcotrione; sulfentrazone (FMC-97285, F-6285); sulfazuron; sulfometuron(-methyl); sulfosate (ICI-A0224); sulfosulfuron; TCA(-sodium); tebutam (GCP-5544); tebuthiuron; tecnacene; tefuryltrione; tembotrione; tepraloxydim; terbacil; terbucarb; terbuchlor; terbumeton; terbuthylazine; terbutryn; TFH 450, i.e. N,N-diethyl-3-[(2-ethyl-6-methylphenyl)-sulfonyl]-1H-1,2,4-triazole-1-carboxamide; thenylchlor (NSK-850); thiafluamide, thiazafluoron; thiazopyr (Mon-13200); thidiazimin (SN-24085); thidiazuron; thiencarbazone(-methyl); thifensulfuron(-methyl); thiobencarb; Ti 35; tiocarbazil; topramezone; tralkoxydim; tri-allate; triasulfuron; triaziflam; triazofenamide; tribenuron(-methyl); triclopyr; tridiphane; trietazine; trifloxysulfuron(-sodium); trifluralin; triflusulfuron and esters (e.g. methyl ester, DPX-66037); trimeturon; trinexapac; tritosulfuron; tsitodef; uniconazole; vernolate; WL 110547, i.e. 5-phenoxy-1-[3-(trifluoromethyl)phenyl]-1H-tetrazole; D-489; ET-751; KIH-218; KIH-485; KIH-509; KPP-300; LS 82-556; NC-324; NC-330; DPX-N8189; SC-0774; DOWCO-535; DK-8910; V-53482; PP-600; MBH-001; TH-547; SYN-523; IDH-100; SYP-249; HOK-201; IR-6396; MTB-951; NC-620.

Of particular interest is the selective control of harmful plants in crops of useful plants and ornamental plants. Although the inventive hydrate and the crystal form I of the sodium salt of 2-iodo-N-[(4-methoxy-6-methyl-1,3,5-triazin-2-yl)carbamoyl]benzenesulfonamide, especially the inventive hydrate, already have very good to adequate selectivity in many crops, it is possible in principle, in some crops and in particular also in the case of mixtures with other herbicides which are less selective, for phytotoxicities to occur in the crop plants. In this regard, combinations of particular interest comprising the inventive hydrate and of the crystal form I of the sodium salt of 2-iodo-N-[(4-methoxy-6-methyl-1,3,5-triazin-2-yl)carbamoyl]benzenesulfonamide, especially the inventive hydrate, are those which comprise the inventive hydrate and the crystal form I of the sodium salt of 2-iodo-N-[(4-methoxy-6-methyl-1,3,5-triazin-2-yl)carbamoyl]benzenesulfonamide, especially the inventive hydrate, or combinations thereof, with other herbicides or pesticides and safeners. The safeners, which are used in an antidotically effective amount, reduce the phytotoxic side effects of the herbicides/pesticides employed, for example in economically important crops, such as cereals (wheat, barley, rye, corn, rice, millet), sugarbeet, sugarcane, oilseed rape, cotton and soybeans, preferably cereals.

The safeners are preferably selected from the group consisting of:

A) compounds of the formula (S-I)

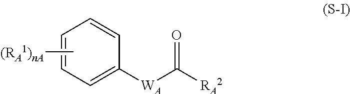

where the symbols and indices are each defined as follows:
n_{A} is a natural number from 0 to 5, preferably 0 to 3;
R_{A}¹ is halogen, (C₁-C₄)-alkyl, (C₁-C₄)-alkoxy, nitro or (C₁-C₄)-haloalkyl;
W_{A} is an unsubstituted or substituted divalent heterocyclic radical from the group of the partially unsaturated or aromatic five-membered heterocycles having 1 to 3 ring heteroatoms of the N or O type, where at least one nitrogen atom and at most one oxygen atom is present in the ring, preferably a radical from the group of (W_{A}¹) to (W_{A}⁴)

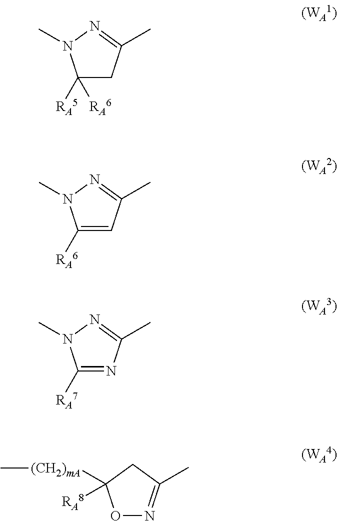

m_{A} is 0 or 1;
R_{A}² is OR_{A}³, SR_{A}³ or NR_{A}³R_{A}⁴ or a saturated or unsaturated 3- to 7-membered heterocycle having at least one nitrogen atom and up to three heteroatoms, preferably from the group consisting of O and S, which is attached via the nitrogen atom to the carbonyl group in (S-I) and which is unsubstituted or substituted by radicals from the group of (C₁-C₄)-alkyl, (C₁-C₄)-alkoxy or optionally substituted phenyl, preferably a radical of the formula OR_{A}³, NHR_{A}⁴ or N(CH₃)₂, especially of the formula OR_{A}³;
R_{A}³ is hydrogen or an unsubstituted or substituted aliphatic hydrocarbyl radical, preferably having a total of 1 to 18 carbon atoms;
R_{A}⁴ is hydrogen, (C₁-C₆)-alkyl, (C₁-C₆)-alkoxy or substituted or unsubstituted phenyl;
R_{A}⁵ is H, (C₁-C₈)-alkyl, (C₁-C₈)-haloalkyl, (C₁-C₄)-alkoxy-(C₁-C₈)-alkyl, cyano or COOR_{A}⁹ in which R_{A}⁹ is hydrogen, (C₁-C₈)-alkyl, (C₁-C₈)-haloalkyl, (C₁-C₄)-alkoxy-(C₁-C₄)-alkyl, (C₁-C₆)-hydroxyalkyl, (C₃-C₁₂)-cycloalkyl or tri-(C₁-C₄)-alkylsilyl;
R_{A}^{E}, R_{A}⁷, R_{A}⁸ are the same or different and are each hydrogen, (C₁-C₈)-alkyl, (C₁-C₈)-haloalkyl, (C₃-C₁₂)-cycloalkyl or substituted or unsubstituted phenyl;
   preferably:
a) compounds of the dichlorophenylpyrazoline-3-carboxylic acid type, preferably compounds such as ethyl 1-(2,4-dichlorophenyl)-5-(ethoxycarbonyl)-5-methyl-2-pyrazoline-3-carboxylate (S1-1) (“mefenpyr-diethyl”, see Pestic. Man.), and related compounds as described in WO 91/07874;
b) derivatives of dichlorophenylpyrazolecarboxylic acid, preferably compounds such as ethyl 1-(2,4-dichlorophenyl)-5-methylpyrazole-3-carboxylate (S1-2), ethyl 1-(2,4-dichlorophenyl)-5-isopropylpyrazole-3-carboxylate (S1-3), ethyl 1-(2,4-dichlorophenyl)-5-(1,1-dimethylethyl)pyrazole-3-carboxylate (S1-4), ethyl 1-(2,4-dichlorophenyl)-5-phenylpyrazole-3-carboxylate (S1-5) and related compounds, as described in EP-A-333 131 and EP-A-269 806;
c) compounds of the triazolecarboxylic acid type, preferably compounds such as fenchlorazole(-ethyl ester), i.e. ethyl 1-(2,4-dichlorophenyl)-5-trichloromethyl-(1H)-1,2,4-triazole-3-carboxylate (S1-6), and related compounds, as described in EP-A-174 562 and EP-A-346 620;
d) compounds of the 5-benzyl- or 5-phenyl-2-isoxazoline-3-carboxylic acid or of the 5,5-diphenyl-2-isoxazoline-3-carboxylic acid type, preferably compounds such as ethyl 5-(2,4-dichlorobenzyl)-2-isoxazoline-3-carboxylate (S1-7) or ethyl 5-phenyl-2-isoxazoline-3-carboxylate (S1-8) and related compounds as described in WO 91/08202, or ethyl 5,5-diphenyl-2-isoxazolinecarboxylate (S1-9) (“isoxadifen-ethyl”) or n-propyl 5,5-diphenyl-2-isoxazolinecarboxylate (S1-10) or ethyl 5-(4-fluorophenyl)-5-phenyl-2-isoxazoline-3-carboxylate (S1-11), as described in patent application WO-A-95/07897.
B) quinoline derivatives of the formula (S-II)

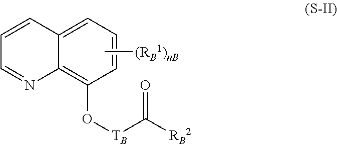

where the symbols and indices are each defined as follows:
R_{B}¹ is halogen, (C₁-C₄)-alkyl, (C₁-C₄)-alkoxy, nitro or (C₁-C₄)-haloalkyl;
n_{B} is a natural number from 0 to 5, preferably 0 to 3;
R_{B}² is OR_{B}³, SR_{B}³ or NR_{B}³R_{B}⁴ or a saturated
or unsaturated 3- to 7-membered heterocycle having at least one nitrogen atom and up to 3 heteroatoms, preferably from the group of O and S, which is attached via the nitrogen atom to the carbonyl group in (S-II) and which is unsubstituted or substituted by radicals from the group of (C₁-C₄)-alkyl, (C₁-C₄)-alkoxy or optionally substituted phenyl, preferably a radical of the formula OR_{B}³, NHR_{B}⁴ or N(CH₃)₂, especially of the formula OR_{B}³;
R_{B}³ is hydrogen or an unsubstituted or substituted aliphatic hydrocarbyl radical, preferably having a total of 1 to 18 carbon atoms;
R_{B}⁴ is hydrogen, (C₁-C₆)-alkyl, (C₁-C₆)-alkoxy or substituted or unsubstituted phenyl;
T_{B} is a (Cᵢ- or C₂)-alkanediyl chain which is unsubstituted or substituted by one or two (C₁-C₄)-alkyl radicals or by [(C₁-C₃)-alkoxy]carbonyl;
   preferably:
a) compounds of the 8-quinolinoxyacetic acid type (S2), preferably 1-methylhexyl (5-chloro-8-quinolinoxy)acetate (common name “cloquintocet-mexyl” (S2-1) (see Pestic. Man.),
1,3-dimethyl-but-1-yl (5-chloro-8-quinolinoxy)acetate (S2-2),
4-allyloxybutyl (5-chloro-8-quinolinoxy)acetate (S2-3),
1-allyloxyprop-2-yl (5-chloro-8-quinolinoxy)acetate (S2-4),
ethyl (5-chloro-8-quinolinoxy)acetate (S2-5),
methyl (5-chloro-8-quinolinoxy)acetate (S2-6),
allyl (5-chloro-8-quinolinoxy)acetate (S2-7),
2-(2-propylideneiminoxy)-1-ethyl (5-chloro-8-quinolinoxy)acetate (S2-8), 2-oxoprop-1-yl
(5-chloro-8-quinolinoxy)acetate (S2-9) and related compounds, as described in EP-A-86 750, EP-A-94 349 and EP-A-191 736 or EP-A-0 492 366, and the hydrates and salts thereof, as described in WO-A-2002/034048.
b) Compounds of the (5-chloro-8-quinolinoxy)malonic acid type, preferably compounds such as diethyl (5-chloro-8-quinolinoxy)malonate, diallyl (5-chloro-8-quinolinoxy)malonate, methyl ethyl (5-chloro-8-quinolinoxy)malonate and related compounds, as described in EP-A-0 582 198.
C) compounds of the formula (S-III)

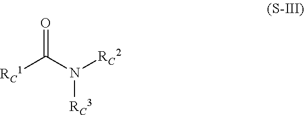

where the symbols and indices are each defined as follows:
R_{c}¹ is (C₁-C₄)-alkyl, (C₁-C₄)-haloalkyl, (C₂-C₄)-alkenyl, (C₂-C₄)-haloalkenyl, (C₃-C₇)-cycloalkyl, preferably dichloromethyl;
R_{c}², R_{c}³ are the same or different and are each hydrogen, (C₁-C₄)-alkyl, (C₂-C₄)-alkenyl, (C₂-C₄)-alkynyl, (C₁-C₄)-haloalkyl, (C₂-C₄)-haloalkenyl, (C₁-C₄)-alkylcarbamoyl-(C₁-C₄)-alkyl, (C₂-C₄)-alkenylcarbamoyl-(C₁-C₄)-alkyl, (C₁-C₄)-alkoxy-(C₁-C₄)-alkyl, dioxolanyl-(C₁-C₄)-alkyl, thiazolyl, furyl, furylalkyl, thienyl, piperidyl, substituted or unsubstituted phenyl, or R_{c}² and R_{c}³ together form a substituted or unsubstituted heterocyclic ring, preferably an oxazolidine, thiazolidine, piperidine, morpholine, hexahydropyrimidine or benzoxazine ring;
   preferably:
active ingredients of the dichloroacetamide type which are frequently employed as pre-emergence safeners (soil-acting safeners), for example “dichlormid” (see Pestic. Man.) (=N,N-diallyl-2,2-dichloroacetamide), “R-29148” (=3-dichloroacetyl-2,2,5-trimethyl-1,3-oxazolidine) from Stauffer,
“R-28725” (=3-dichloroacetyl-2,2-dimethyl-1,3-oxazolidine) from Stauffer, “benoxacor” (see Pestic. Man.) (=4-dichloroacetyl-3,4-dihydro-3-methyl-2H-1,4-benzoxazine),
“PPG-1292” (=N-allyl-N-[(1,3-dioxolan-2-yl)methyl]dichloroacetamide) from PPG Industries,
“DKA-24” (=N-allyl-N-[(allylaminocarbonyl)methyl]dichloroacetamide) from Sagro-Chem,
“AD-67” or “MON 4660” (=3-dichloroacetyl-1-oxa-3-azaspiro[4,5]decane) from Nitrokemia or Monsanto,
“TI-35” (=1-dichloroacetylazepane) from TRI-Chemical RT “diclonon” (dicyclonone) or “BAS145138” or “LAB145138” (=3-dichloroacetyl-2,5,5-trimethyl-1,3-diazabicyclo[4.3.0]nonane) from BASF and
“furilazole” or “MON 13900” (see Pestic. Man.) (=(RS)-3-dichloroacetyl-5-(2-furyl)-2,2-dimethyloxazolidine).
D) N-acylsulfonamides of the formula (S-IV) and salts thereof

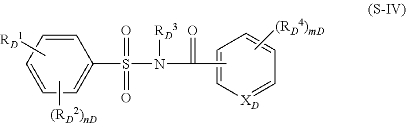

in which
X_{D} is CH or N;
R_{D}¹ is CO—NR_{D}⁵R_{D}⁶ or NHCO—R_{D}⁷;
R_{D}² is halogen, (C₁-C₄)-haloalkyl, (C₁-C₄)-haloalkoxy, nitro, (C₁-C₄)-alkyl, (C₁-C₄)-alkoxy, (C₁-C₄)-alkylsulfonyl, (C₁-C₄)-alkoxycarbonyl or (C₁-C₄)-alkylcarbonyl;
R_{D}³ is hydrogen, (C₁-C₄)-alkyl, (C₂-C₄)-alkenyl or (C₂-C₄)-alkynyl;
R_{D}⁴ is halogen, nitro, (C₁-C₄)-alkyl, (C₁-C₄)-haloalkyl, (C₁-C₄)-haloalkoxy, (C₃-C₆)-cycloalkyl, phenyl, (C₁-C₄)-alkoxy, cyano, (C₁-C₄)-alkylthio, (C₁-C₄)-alkylsulfinyl, (C₁-C₄)-alkylsulfonyl, (C₁-C₄)-alkoxycarbonyl or (C₁-C₄)-alkylcarbonyl;
R_{D}⁵ is hydrogen, (C₁-C₆)-alkyl, (C₃-C₆)-cycloalkyl, (C₂-C₆)-alkenyl, (C₂-C₆)-alkynyl, (C₅-C₆)-cycloalkenyl, phenyl or 3- to 6-membered heterocyclyl containing v_{D} heteroatoms from the group of nitrogen, oxygen and sulfur, where the seven latter radicals are substituted by v_{D} substituents from the group of halogen, (C₁-C₆)-alkoxy, (C₁-C₆)-haloalkoxy, (C₁-C₂)-alkylsulfinyl, (C₁-C₂)-alkylsulfonyl, (C₃-C₆)-cycloalkyl, (C₁-C₄)-alkoxycarbonyl, (C₁-C₄)-alkylcarbonyl and phenyl and, in the case of cyclic radicals, also (C₁-C₄)-alkyl and (C₁-C₄)-haloalkyl;
R_{D}⁶ is hydrogen, (C₁-C₆)-alkyl, (C₂-C₆)-alkenyl or (C₂-C₆)-alkynyl, where the three latter radicals are substituted by v_{D} radicals from the group of halogen, hydroxy, (C₁-C₄)-alkyl, (C₁-C₄)-alkoxy and (C₁-C₄)-alkylthio, or
R_{D}⁵ and R_{D}⁶ together with the nitrogen atom bearing them form a pyrrolidinyl or piperidinyl radical;
R_{D}⁷ is hydrogen, (C₁-C₄)-alkylamino, di-(C₁-C₄)-alkylamino, (C₁-C₆)-alkyl, (C₃-C₆)-cycloalkyl, where the 2 latter radicals are substituted by v_{D} substituents from the group of halogen, (C₁-C₄)-alkoxy, halo-(C₁-C₆)-alkoxy and (C₁-C₄)-alkylthio and, in the case of cyclic radicals, also (C₁-C₄)-alkyl and (C₁-C₄)-haloalkyl;
n_{D} is 0, 1 or 2;
m_{D} is 1 or 2;
v_{D} is 0, 1, 2 or 3;
   among these, preference is given to compounds of the N-acylsulfonamide type, for example of the following formula (S-V), which are known, for example, from WO 97/45016:

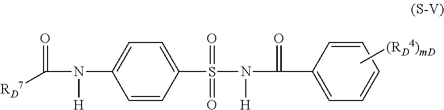

in which
R_{D}⁷ is (C₁-C₆)-alkyl, (C₃-C₆)-cycloalkyl, where the 2 latter radicals are substituted by v_{D} substituents from the group consisting of halogen, (C₁-C₄)-alkoxy, halo-(C₁-C₆)-alkoxy and (C₁-C₄)-alkylthio and, in the case of cyclic radicals, also (C₁-C₄)-alkyl and (C₁-C₄)-haloalkyl;
R_{D}⁴ is halogen, (C₁-C₄)-alkyl, (C₁-C₄)-alkoxy, CF₃,
m_{D} is 1 or 2;
v_{D} is 0, 1, 2 or 3;
   and
   acylsulfamoylbenzamides, for example of the following formula (S-VI), which are known, for example, from WO 99/16744:

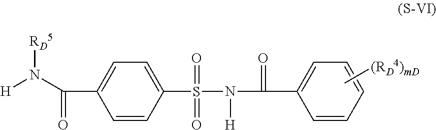

for example those in which
R_{D}⁵=cyclopropyl and (R_{D}⁴)=2-OMe (“cyprosulfamide”, S3-1),
R_{D}⁵=cyclopropyl and (R_{D}⁴)=5-Cl-2-OMe (S3-2),
R_{D}⁵=ethyl and (R_{D}⁴)=2-OMe (S3-3),
R_{D}⁵=isopropyl and (R_{D}⁴)=5-C₁₋₂-OMe (S3-4) and
R_{D}⁵=isopropyl and (R_{D}⁴)=2-OMe (S3-5);
   and
   compounds of the N-acylsulfamoylphenylurea type of the formula (S-VII), which are known, for example, from EP-A-365484,

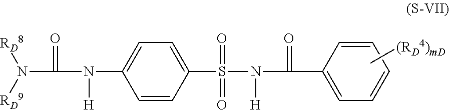

in which
R_{D}⁸ and R_{D}⁹ are each independently hydrogen, (C₁-C₈)-alkyl, (C₃-C₈)-cycloalkyl, (C₃-C₆)-alkenyl, (C₃-C₆)-alkynyl,
R_{D}⁴ is halogen, (C₁-C₄)-alkyl, (C₁-C₄)-alkoxy, CF₃
m_{D} is 1 or 2;
   among these, especially
1-[4-(N-2-methoxybenzoylsulfamoyl)phenyl]-3-methylurea,
1-[4-(N-2-methoxybenzoylsulfamoyl)phenyl]-3,3-dimethylurea,
1-[4-(N-4,5-dimethylbenzoylsulfamoyl)phenyl]-3-methylurea,
1-[4-(N-naphthoylsulfamoyl)phenyl]-3,3-dimethylurea,
G) active ingredients from the class of the hydroxyaromatics and aromatic-aliphatic carboxylic acid derivatives, for example
ethyl 3,4,5-triacetoxybenzoate, 3,5-dimethoxy-4-hydroxybenzoic acid, 3,5-dihydroxybenzoic acid, 4-hydroxysalicylic acid, 4-fluorosalicyclic acid, 1,2-dihydro-2-oxo-6-trifluoromethylpyridine-3-carboxamide, 2-hydroxycinnamic acid, 2,4-dichlorocinnamic acid, as described in WO 2004084631, WO 2005015994, WO 2006007981, WO 2005016001;
H) active ingredients from the class of the 1,2-dihydroquinoxalin-2-ones, for example 1-methyl-3-(2-thienyl)-1,2-dihydroquinoxalin-2-one, 1-methyl-3-(2-thienyl)-1,2-dihydroquinoxaline-2-thione, 1-(2-aminoethyl)-3-(2-thienyl)-1,2-dihydroquinoxalin-2-one hydrochloride, 1-(2-methylsulfonylaminoethyl)-3-(2-thienyl)-1,2-dihydroquinoxalin-2-one, as described in WO 2005112630,
I) active ingredients which, in addition to herbicidal action against harmful plants, also have safener action in crop plants such as rice, for example
“dimepiperate” or “MY-93” (see Pestic. Man.) (═S-1-methyl-1-phenylethyl piperidine-1-thiocarboxylate), which is known as a safener for rice against damage by the herbicide molinate,
“daimuron” or “SK 23” (see Pestic. Man.) (=1-(1-methyl-1-phenylethyl)-3-p-tolyl-urea), which is known as a safener for rice against damage by the herbicide imazosulfuron,
“cumyluron”=“JC-940” (=3-(2-chlorophenylmethyl)-1-(1-methyl-1-phenylethyl)urea, see JP-A-60087254), which is known as a safener for rice against damage by some herbicides,
“methoxyphenon” or “NK 049” (=3,3′-dimethyl-4-methoxybenzophenone), which is known as a safener for rice against damage by some herbicides,
“CSB” (=1-bromo-4-(chloromethylsulfonyl)benzene) (CAS reg. no. 54091-06-4 from Kumiai), which is known as a safener for rice against damage by some herbicides,
K) compounds of the formula (S-IX),
   as described in WO-A-1998/38856,

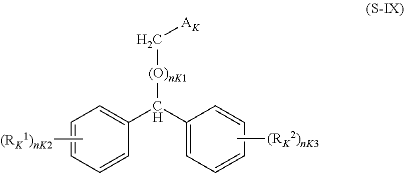

where the symbols and indices are each defined as follows:
R_{K}¹, R_{K}² are each independently halogen, (C₁-C₄)-alkyl, (C₁-C₄)-alkoxy, (C₁-C₄)-haloalkyl, (C₁-C₄)-alkylamino, di-(C₁-C₄)-alkylamino, nitro;
A_{K} is COOR_{K}³ or COOR_{K}⁴
R_{K}³, R_{K}⁴ are each independently hydrogen, (C₁-C₄)-alkyl, (C₂-C₆)-alkenyl, (C₂-C₄)-alkynyl, cyanoalkyl, (C₁-C₄)-haloalkyl, phenyl, nitrophenyl, benzyl, halobenzyl, pyridinylalkyl and alkylammonium,
n_{K}¹ is 0 or 1
n_{K}², n_{K}³ are each independently 0, 1 or 2,
preferably: methyl (diphenylmethoxy)acetate (CAS reg. no.: 41858-19-9),
L) compounds of the formula (S-X),
   as described in WO A-98/27049,

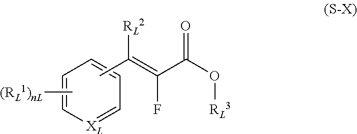

where the symbols and indices are each defined as follows:
X_{L} is CH or N,
n_{L} in the case that X═N is an integer from 0 to 4 and
   in the case that X═CH is an integer from 0 to 5,
R_{L}¹ is halogen, (C₁-C₄)-alkyl, (C₁-C₄)-haloalkyl, (C₁-C₄)-alkoxy, (C₁-C₄)-haloalkoxy, nitro, (C₁-C₄)-alkylthio, (C₁-C₄)-alkylsulfonyl, (C₁-C₄)-alkoxycarbonyl, optionally substituted phenyl, optionally substituted phenoxy,
R_{L}2 is hydrogen or (C₁-C₄)-alkyl,
R_{L}³ is hydrogen, (C₁-C₈)-alkyl, (C₂-C₄)-alkenyl, (C₂-C₄)-alkynyl or aryl, where each of the aforementioned carbon-containing radicals is unsubstituted or substituted by one or more, preferably by up to three, identical or different radicals from the group consisting of halogen and alkoxy; or salts thereof,
M) active ingredients from the class of the 3-(5-tetrazolylcarbonyl)-2-quinolones, e.g. 1,2-dihydro-4-hydroxy-1-ethyl-3-(5-tetrazolylcarbonyl)-2-quinolone (CAS reg. no.: 219479-18-2), 1,2-dihydro-4-hydroxy-1-methyl-3-(5-tetrazolylcarbonyl)-2-quinolone (CAS reg. no.: 95855-00-8), as described in WO-A-1999000020;
N) compounds of the formula (S-XI) or (S-XII),
   as described in WO-A-2007023719 and WO-A-2007023764,

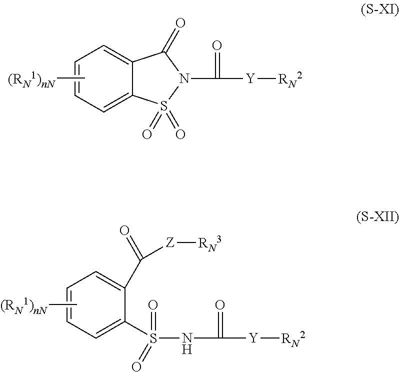

in which
R_{N}¹ is halogen, (C₁-C₄)-alkyl, methoxy, nitro, cyano, CF₃, OCF₃
Y, Z are each independently O or S,
n_{N} is an integer from 0 to 4,
R_{N}² is (C₁-C₁₆)-alkyl, (C₂-C₆)-alkenyl, (C₃-C₆)-cycloalkyl, aryl; benzyl, halobenzyl,
R_{N}³ is hydrogen, (C₁-C₆)-alkyl;
O) one or more compounds from the group of:
1,8-naphthalic anhydride,
O,O-diethyl S-2-ethylthioethyl phosphordithioate (disulfoton),
4-chlorophenyl methylcarbamate (mephenate),
O,O-diethyl O-phenylphosphorothioate (dietholate),
4-carboxy-3,4-dihydro-2H-1-benzopyran-4-acetic acid (CL-304415, CAS reg. no.: 31541-57-8),
2-propenyl 1-oxa-4-azaspiro[4.5]decane-4-carbodithioate (MG-838, CAS reg. no.: 133993-74-5),
methyl [(3-oxo-1H-2-benzothiopyran-4(3H)-ylidene)methoxy]acetate (from WO-A-98/13361; CAS reg. no.: 205121-04-6),
cyanomethoxyimino(phenyl)acetonitrile (cyometrinil),
1,3-dioxolan-2-ylmethoxyimino(phenyl)acetonitrile (oxabetrinil),
4′-chloro-2,2,2-trifluoroacetophenone 0-1,3-dioxolan-2-ylmethyl oxime (fluxofenim),
4,6-dichloro-2-phenylpyrimidine (fenclorim),
benzyl 2-chloro-4-trifluoromethyl-1,3-thiazole-5-carboxylate (flurazole),
2-dichloromethyl-2-methyl-1,3-dioxolane (MG-191),
including the stereoisomers and the salts commonly used in agriculture.

A mixture with other known active ingredients, such as fungicides, insecticides, acaricides, nematicides, bird repellents, plant nutrients and soil structure improvers, is also possible.

Some of the safeners are already known as herbicides and thus, in addition to the herbicidal action against harmful plants, at the same time also display protective action on the crop plants.

The weight ratios of herbicide (mixture) to safener depends generally on the application rate of the herbicide and the efficacy of the particular safener and can vary within wide limits, for example in the range from 200:1 to 1:200, preferably 100:1 to 1:100, especially 20:1 to 1:20. The safeners, analogously to the inventive hydrate and/or the crystal form I of the sodium salt of 2-iodo-N-[(4-methoxy-6-methyl-1,3,5-triazin-2-yl)carbamoyl]benzenesulfonamide or mixtures thereof, can be formulated with further herbicides/pesticides, and provided and employed as a finished formulation or tankmix with the herbicides.

The outside conditions, including temperature, humidity, the type of herbicide used, among others, cause variation in the required application rate of the inventive hydrate and of the crystal form I of the sodium salt of 2-iodo-N-[(4-methoxy-6-methyl-1,3,5-triazin-2-yl)carbamoyl]benzenesulfonamide. It can vary within wide limits, for example between 0.001 and 10 000 g/ha or more of active substance, but it is preferably between 0.5 and 5000 g/ha, more preferably between 0.5 and 1000 g/ha and most preferably between 0.5 and 500 g/ha.

The inventive hydrate and the crystal form I of the sodium salt of 2-iodo-N-[(4-methoxy-6-methyl-1,3,5-triazin-2-yl)carbamoyl]benzenesulfonamide can be used, for example, with the following plants:

Dicotyledonous weeds of the genera: Abutilon, Amaranthus, Ambrosia, Anoda, Anthemis, Aphanes, Atriplex, Bellis, Bidens, Capsella, Carduus, Cassia, Centaurea, Chenopodium, Cirsium, Convolvulus, Datura, Desmodium, Emex, Erysimum, Euphorbia, Galeopsis, Galinsoga, Hibiscus, Ipomoea, Kochia, Lamium, Lepidium, Lindernia, Matricaria, Mentha, Mercurialis, Mullugo, Myosotis, Papaver, Pharbitis, Plantago, Polygonum, Portulaca, Ranunculus, Raphanus, Rorippa, Rotala, Rumex, Salsola, Senecio, Sesbania, Sida, Sinapis, Solanum, Sonchus, Sphenoclea, Stellaria, Taraxacum, Thlaspi, Trifolium, Urtica, Veronica, Viola, Xanthium.

Dicotyledonous crops of the genera: Arachis, Cucumis, Cucurbita, Daucus, Glycine, Gossypium, Linum, Lycopersicon, Nicotiana, Pisum, Solanum, Vicia.

Monocotyledonous weeds of the genera: Aegilops, Agropyron, Agrostis, Alopecurus, Apera, Avena, Brachiaria, Bromus, Cenchrus, Commelina, Cynodon, Cyperus, Dactyloctenium, Digitaria, Echinochloa, Eleocharis, Eleusine, Eragrostis, Eriochloa, Festuca, Fimbristylis, Heteranthera, Imperata, Ischaemum, Leptochloa, Lolium, Monochoria, Panicum, Paspalum, Phalaris, Phleum, Poa, Rottboellia, Sagittaria, Scirpus, Setaria, Sorghum.

Monocotyledonous crops of the genera: Allium, Ananas, Asparagus, Avena, Hordeum, Oryza, Panicum, Saccharum, Secale, Sorghum, Triticale, Triticum, Zea.

The use of the inventive hydrate and of the crystal form I of the sodium salt of 2-iodo-N-[(4-methoxy-6-methyl-1,3,5-triazin-2-yl)carbamoyl]benzenesulfonamide is, however, in no way restricted to these genera, but also extends in the same way to other plants.

The inventive hydrate and the crystal form I of the sodium salt of 2-iodo-N-[(4-methoxy-6-methyl-1,3,5-triazin-2-yl)carbamoyl]benzenesulfonamide, especially the inventive hydrate, are also suitable, depending on the concentration, for total weed control, for example on industrial and railroad sites, and on paths and open spaces with and without planted trees. The inventive hydrate and/or the crystal form I of the sodium salt of 2-iodo-N-[(4-methoxy-6-methyl-1,3,5-triazin-2-yl)carbamoyl]benzenesulfonamide can likewise be used for weed control in perennial crops, for example forests, decorative tree plantings, orchards, vineyards, citrus groves, nut orchards, banana plantations, coffee plantations, tea plantations, rubber plantations, oil palm plantations, cocoa plantations, soft fruit plantings and hop fields, on lawns, turf and pastureland, and for selective control of weeds in annual crops.

The inventive hydrate and the crystal form I of the sodium salt of 2-iodo-N-[(4-methoxy-6-methyl-1,3,5-triazin-2-yl)carbamoyl]benzenesulfonamide, especially the inventive hydrate, show strong herbicidal efficacy and a broad spectrum of action on application to the soil and to exposed plant parts. They are also suitable to a certain extent for selective control of monocotyledonous and dicotyledonous weeds in monocotyledonous and dicotyledonous crops, both by the pre-emergence and by the post-emergence method, or in sequential application.

The inventive hydrate and the crystal form I of the sodium salt of 2-iodo-N-[(4-methoxy-6-methyl-1,3,5-triazin-2-yl)carbamoyl]benzenesulfonamide, especially the inventive hydrate, show a favorable influence on subsequent crops (rotation behavior), i.e. zero or an extremely low phytotoxicity (for example in the form of (a) light green to yellow leaf veins, (b) yellowing of entire plants, (c) retarded plant growth, (d) abnormal development of younger plant parts or of the entire plant) on various subsequent crops which are sensitive to the inventive hydrate and to the crystal form I of the sodium salt of 2-iodo-N-[(4-methoxy-6-methyl-1,3,5-triazin-2-yl)carbamoyl]benzenesulfonamide, for example sugarbeet, sunflower or crucifers, such as oilseed rape, mustard and field mustard.

The preparation and the use of the inventive hydrate and of the crystal form I of the sodium salt of 2-iodo-N-[(4-methoxy-6-methyl-1,3,5-triazin-2-yl)carbamoyl]benzenesulfonamide are evident from the examples which follow.

### Working examples

A Preparation of the inventive hydrate of the sodium salt of 2-iodo-N-[(4-methoxy-6-methyl-1,3,5-triazin-2-yl)carbamoyl]benzenesulfonamide:

### Example 1

1000 g of the amorphous sodium salt of 2-iodo-N-[(4-methoxy-6-methyl-1,3,5-triazin-2-yl)carbamoyl]benzenesulfonamide were suspended in 3000 ml of an acetonitrile/water mixture (95:5% by vol.) and stirred at room temperature for 3.5 h. Subsequently, the suspension was sucked through a suction filter and the filtercake was dried under reduced pressure at 45-70° C.

### Example 2

2 g of the amorphous sodium salt of 2-iodo-N-[(4-methoxy-6-methyl-1,3,5-triazin-2-yl)carbamoyl]benzenesulfonamide were dissolved completely in boiling acetone (56° C.) and then cooled gradually to room temperature, i.e. with a cooling rate of less than 20° C./h. The crystals are filtered off and dried at temperatures of <60° C.

B Preparation of the inventive crystal form I of the sodium salt of 2-iodo-N-[(4-methoxy-6-methyl-1,3,5-triazin-2-yl)carbamoyl]benzenesulfonamide:

0.5 g of the amorphous sodium salt of 2-iodo-N-[(4-methoxy-6-methyl-1,3,5-triazin-2-yl)carbamoyl]benzenesulfonamide were suspended in 5 ml of ethanol and stirred at 60-80° C. for 5 h. Subsequently, the suspension was filtered and dried.

### Stability Tests

The oil dispersions described were produced analogously to DE 19951427. An oil dispersion of the inventive hydrate of the sodium salt of 2-iodo-N-[(4-methoxy-6-methyl-1,3,5-triazin-2-yl)carbamoyl]benzenesulfonamide, compared to an oil dispersion of the amorphous sodium salt of 2-iodo-N-[(4-methoxy-6-methyl-1,3,5-triazin-2-yl)carbamoyl]benzenesulfonamide, even after storage at different temperatures for several weeks, did not exhibit any signs of precipitation or crystallization of the active ingredient. Nor was any change found in the viscosity of the oil dispersions. In the case of use of the amorphous sodium salt of 2-iodo-N-[(4-methoxy-6-methyl-1,3,5-triazin-2-yl)carbamoyl]benzenesulfonamide, in contrast, even in the course of production of oil dispersions or in the course of storage thereof at room temperature, there are significant rises in viscosity, up to and including complete solidification, or precipitation or crystallization of the active ingredient, within hours or a few days.

1. A sodium salt of 2-iodo-N-[(4-methoxy-6-methyl-1,3,5-triazin-2-yl)carbamoyl]benzenesulfonamide, wherein the salt is present as crystal form I or hydrate (crystal form II), the respective x-ray powder diffractograms of which at 25° C. have at least three of the following 20 (2 theta) values:

| | |
|---|---|
| 2 θ | D [Å ± 0.2] |

crystal form I

| | |
|---|---|
| 6.0 | 14.7 |
| 8.9 | 9.9 |
| 9.9 | 9.0 |
| 11.1 | 8.0 |
| 12.8 | 6.9 |
| 16.7 | 5.3 |
| 24.0 | 3.7 |
| 24.3 | 3.7 |
| 26.6 | 3.4 |
| 26.9 | 3.3 |

hydrate (crystal form II)

| | |
|---|---|
| 6.8 | 13.0 |
| 7.2 | 12.3 |
| 9.8 | 9.0 |
| 11.8 | 7.5 |
| 13.6 | 6.5 |
| 14.4 | 6.2 |
| 16.6 | 5.3 |
| 22.1 | 4.0 |
| 25.1 | 3.5 |
| 26.4 | 3.4 |

2. The sodium salt as claimed in claim 1, wherein the x-ray powder diffractogram at 25° C. for the hydrate and for the crystal form I has at least four of the 20 values mentioned in claim 1.

3. A composition comprising the sodium salt as claimed in claim 1 and at least one suitable assistant.

4. A crop protection composition for control of mono- and dicotyledonous harmful plants comprising a sodium salt of claim 1.

5. A crop protection composition with growth-regulatory properties in crop plants comprising a sodium salt of claim 1.

6. The composition as claimed in claim 5, which is a crop protection composition for control of harmful plants in crops of known plants or of genetically modified plants yet to be developed.

7. The composition as claimed claim 4, which is adapted for application of crop protection compositions for control of harmful plants in economically important transgenic crops of useful and ornamental plants and/or crops thereof obtained by mutant selection.

8. A process for preparing the sodium salt of 2-iodo-N-[(4-methoxy-6-methyl-1,3,5-triazin-2-yl)carbamoyl]benzenesulfonamide as defined in claim 1, which comprises the following steps:
(a) dissolving and/or suspending the sodium salt of 2-iodo-N-[(4-methoxy-6-methyl-1,3,5-triazin-2-yl)carbamoyl]benzenesulfonamide in a solvent or solvent mixture;
(b) heating the solution and/or suspension to at least 30° C.;
(c) gradually cooling the solution and/or the suspension.

9. The process as claimed in claim 8, in which the gradual cooling is undertaken stepwise.

10. The process as claimed in claim 8, in which the solvent or solvent mixture used is selected from the group consisting of acetonitrile, acetonitrile/water mixtures, water-miscible solvents, preferably lower alcohols such as methanol, ethanol, 2-propanol, or ketones, preferably acetone, 2-butanone and methyl isobutyl ketone.

11. The sodium salt as claimed in claim 1, in which the crystal form I and the hydrate have an infrared spectrum according to the table below and where the particular band maxima are reported in cm⁻¹:

| | |
|---|---|
| Crystal | |
| form I | Hydrate |
| | | | |
|---|---|---|---|
| 3364 | 966 | 1648 | 706 |
| 1637 | 892 | 1560 | 698 |
| 1624 | 880 | 1519 | 640 |
| 1562 | 868 | 1474 | 628 |
| 1508 | 818 | 1446 | 586 |
| 1471 | 811 | 1392 | 572 |
| 1445 | 785 | 1365 | 554 |
| 1395 | 767 | 1346 | |
| 1369 | 754 | 1242 | |
| 1348 | 737 | 1208 | |
| 1279 | 705 | 1140 | |
| 1254 | 695 | 1113 | |
| 1243 | 640 | 1080 | |
| 1209 | 627 | 1036 | |
| 1166 | 584 | 1012 | |
| 1136 | 579 | 992 | |
| 1113 | 568 | 957 | |
| 1096 | 553 | 877 | |
| 1080 | | 811 | |
| 1037 | | 787 | |
| 1011 | | 761 | |
| 992 | | 735 | |

12. The sodium salt as claimed in claim 1, in which the crystal form I and the hydrate have a Raman spectrum according to the table below and where the particular band maxima are reported in cm⁻¹:

| Crystal form I | Hydrate |
|---|---|
| 3127 | 3125 |
| 3060 | 3060 |
| 2930 | 3006 |
| 1650 | 2962 |
| 1633 | 2925 |
| 1571 | 1664 |
| 1562 | 1572 |
| 1517 | 1562 |
| 1446 | 1522 |
| 1397 | 1445 |
| 1368 | 1396 |
| 1342 | 1370 |
| 1273 | 1345 |
| 1245 | 1276 |
| 1186 | 1183 |
| 1163 | 1162 |
| 1136 | 1145 |
| 1113 | 1113 |
| 1096 | 1087 |
| 1081 | 1036 |
| 1040 | 1016 |
| 1017 | 992 |
| 991 | 879 |
| 891 | 788 |
| 879 | 740 |
| 870 | 641 |
| 776 | 587 |
| 737 | 556 |
| 696 | 539 |
| 589 | 388 |
| 579 | 373 |
| 567 | 352 |
| 554 | 342 |
| 540 | 244 |
| 513 | 205 |
| 437 | 131 |
| 371 | 112 |
| 341 | |
| 254 | |
| 245 | |
| 203 | |
| 176 | |
| 137 | |
| 120 | |
